SEQUENCE LISTING (22 sequences)

SEQ ID NO: 1 - DNA, 21 bp, synthetic construct
  source 1..21: mol_type = other DNA
gcgcgaagttatcctacgaat

SEQ ID NO: 2 - DNA, 20 bp, synthetic construct
  source 1..20: mol_type = other DNA
tggtgggttacaggactaca

SEQ ID NO: 3 - DNA, 21 bp, synthetic construct
  source 1..21: mol_type = other DNA
gtagtcctgtaacccaccatc

SEQ ID NO: 4 - DNA, 20 bp, synthetic construct
  source 1..20: mol_type = other DNA
acaggtacgctcagcagaat

SEQ ID NO: 5 - DNA, 21 bp, synthetic construct
  source 1..21: mol_type = other DNA
gcgcgaagttatcctacgaat

SEQ ID NO: 6 - DNA, 18 bp, synthetic construct
  source 1..18: mol_type = other DNA
cccgtttaggctttcgct

SEQ ID NO: 7 - DNA, 18 bp, synthetic construct
  source 1..18: mol_type = other DNA
gcagcgaaagcctaaacg

SEQ ID NO: 8 - DNA, 20 bp, synthetic construct
  source 1..20: mol_type = other DNA
tggtgggttacaggactaca

SEQ ID NO: 9 - DNA, 42 bp, synthetic construct
  source 1..42: mol_type = other DNA
agctagcaataattttgtttaactttaagaaggagatatacc

SEQ ID NO: 10 - DNA, 28 bp, synthetic construct
  source 1..28: mol_type = other DNA
tctagagattaaagaggagaaatactag

SEQ ID NO: 11 - DNA, 49 bp, synthetic construct
  source 1..49: mol_type = other DNA
taacaccgtgcgtgttgacaattttacctctggcggtgataatggttgc

SEQ ID NO: 12 - DNA, 41 bp, synthetic construct
  source 1..41: mol_type = other DNA
ccgagctcgagcaaagcccgccgaaaggcgggcttttctgt

SEQ ID NO: 13 - DNA, 1023 bp, synthetic construct
  source 1..1023: mol_type = other DNA
gcgcgaagttatcctacgaattcgtcaaagctttaacaccgtgcgtgttgacaattttacctctggcggtgataatggttgcgatatcagctagcaataattttgtttaactttaagaaggagatataccatgcttgatctgaaattgggaggcttggctgctgcttgtttgctggtctgttctacggcacttgccgcacctttaccagacacccctggggcgccttttccagcagttgctaacttcgatcgttctggaccttacgctactagcaatcaatccgaaggtccctcatgtcgcatctaccgtccctctaatttgggacaggggggtgtgcgtcacccagtaattttgtggggaaacggtacgggcacaggtccctccacctatgcgggccttttatcccattgggcctcgcatggcttcgtcgtagcggcggctgagacgtcgaatgccggtacagggcgcgaaatgctggcctgtttggactatcttgtgcgcgaaaatgataatccctatgggacttatgctggtaagttgaacacaggacgtgtggggacatccggtcactcccaaggcggcggcggttcaattatggcggggcaggatacccgtgtgcgtacgacggcaccaattcaaccttacaccattggactgggccatgatagcgcctcacaacgtcgccaacaggggccaatgttccttatgtccggtggcggcgacactatcgctatcccctatcttaacgcgcagccagtgtatcttcgcgccaatgtacccgttttctggggcgagcgccgttacgtttctcatttcgagcccgtgggagacggtggtgcatatcgtggccccagcacggcctggtttcgtttccagttgatggacgatcagtcagcgcgtggtactttttatgggactctgtgctccctgtgttcctcattattgtggagtgtcgagcgccgtggcttctagagatgaatacgtataccaggtgtcctgtgagcagcgaaagcctaaacggg

SEQ ID NO: 14 - DNA, 1134 bp, synthetic construct
  source 1..1134: mol_type = other DNA
gcgcgaagttatcctacgaattcgtcaaagctttaacaccgtgcgtgttgacaattttacctctggcggtgataatggttgcgatatcagctagcaataattttgtttaactttaagaaggagatataccatgaagaagaacaaaacccttttggcattgtgcctgggcacgggtttattagcatcgggtcaaacacaggctttctggttcggatcgagcggctatacgcagactaagtacccgatcgtgttgggccacggtatgcttggattcgattcgattctgggggtagactattggtacggaattccggccgccttgcgccgtgacggcgcttctgtctatgtaaccgaggtctctcaattggacacaagcgaggctcgtggcgaacaattattacagcaagtcgaagacattgtagccatctcaggcaaaggcaaagtcaacttaatcggccacagtcacgggggaccaaccactcgctacgtggccgcggtacgcccagacttggttgcgtcagtaacgtctgttggagccccgcataaaggatccgcaacggccgactttcttaaagggatcagtgaaggtccagctggcccagtcgctactccagtattggtaggcattgtaaatgggctgggcgcactgattaacttcctttcaggatcgagctcaacgacgccccaaaacgcgttaggatccttagaatctcttaattccgaaggtgcagcacgctttaatgctaagtttccgcagggcattcctacctctgcatgcggagaaggcgcatattctgtgaacggcgtccgttattactcatggtcagggacaagtcccctgacgaacgtactggacccctcggacttgttattaggcgcatcgagtttaacattcgggagcgaggcaaacgatggcttagtaggtcgttgcagcagccgtatgggccaagtgattcgcgacaactaccgtatgaaccatcttgatgaagtgaaccagacattgggtcttacgagtctgttcgagacagacccggtgacggtttaccgccaacatgcaaaccgtcttaaaaacgctggcctgtagagatgaatacgtataccaggtgtcctgtgagcagcgaaagcctaaacggg

SEQ ID NO: 15 - DNA, 952 bp, synthetic construct
  source 1..952: mol_type = other DNA
gcgcgaagttatcctacgaattcgtcaaagctttaacaccgtgcgtgttgacaattttacctctggcggtgataatggttgcgatatcagctagcaataattttgtttaactttaagaaggagatataccatgagcaaaggagaagaacttttcactggagttgtcccaattcttgttgaattagatggtgatgttaatgggcacaaattttctgtccgtggagagggtgaaggtgatgctacaaacggaaaactcacccttaaatttatttgcactactggaaaactacctgttccgtggccaacacttgtcactactctgacctatggtgttcaatgcttttcccgttatccggatcacatgaaacgtcatgactttttcaagagtgccatgcctgaaggttatgtacaggaacgcactatatctttcaaagatgacgggacctacaagacgcgtgctgaagtcaagtttgaaggtgatacccttgttaatcgtatcgagttaaagggtattgattttaaagaagatggaaacattcttggacacaaactcgagtacaactttaactcacacaatgtatacatcacggcagacaaacaaaagaatggaatcaaagctaacttcaaaattcgccacaacgttgaagatggttccgttcaactagcagaccattatcaacaaaatactccaattggcgatggccctgtccttttaccagacaaccattacctgtcgacacaatctgtcctttcgaaagatcctaacgaaaagcgtgaccacatggtccttcttgagtttgtaactgctgctgggattacacatggcatggatgagctctacaaaccgagctcgagcaaagcccgccgaaaggcgggcttttctgtgggcatgctgagcgatatcaagcttgcggccgcacccagcagcaggatgtagtcctgtaacccacca

SEQ ID NO: 16 - DNA, 1047 bp, synthetic construct
  source 1..1047: mol_type = other DNA
gcgcgaagttatcctacgaattcgtcaaagcttaaaataacatcatcattacgtcgcactgtggcggctatcgcactttaacgtttcgtgctgccccctcagtctatgcaatagaccataaactgcaaaaaaaagtccgctgataaggcttgaaaagttcatttccagacccatttttacatcggctagcaataattttgtttaactttaagaaggagatataccatgagcaaaggagaagaacttttcactggagttgtcccaattcttgttgaattagatggtgatgttaatgggcacaaattttctgtccgtggagagggtgaaggtgatgctacaaacggaaaactcacccttaaatttatttgcactactggaaaactacctgttccgtggccaacacttgtcactactctgacctatggtgttcaatgcttttcccgttatccggatcacatgaaacgtcatgactttttcaagagtgccatgcctgaaggttatgtacaggaacgcactatatctttcaaagatgacgggacctacaagacgcgtgctgaagtcaagtttgaaggtgatacccttgttaatcgtatcgagttaaagggtattgattttaaagaagatggaaacattcttggacacaaactcgagtacaactttaactcacacaatgtatacatcacggcagacaaacaaaagaatggaatcaaagctaacttcaaaattcgccacaacgttgaagatggttccgttcaactagcagaccattatcaacaaaatactccaattggcgatggccctgtccttttaccagacaaccattacctgtcgacacaatctgtcctttcgaaagatcctaacgaaaagcgtgaccacatggtccttcttgagtttgtaactgctgctgggattacacatggcatggatgagctctacaaaccgagctcgagcaaagcccgccgaaaggcgggcttttctgtgggcatgctgagcgatatcaagcttgcggccgcacccagcagcaggatgtagtcctgtaacccacca

SEQ ID NO: 17 - DNA, 1015 bp, synthetic construct
  source 1..1015: mol_type = other DNA
gcgcgaagttatcctacgaattcgtcaaagctttgcttttttctgcaacaatttagcgttttttcccaccatagccaaccgccataacggttggctgttcttcgttgcaaatggcgacccccgtcacactgtctatacttacatgtctgtaaagcgcgttctgcgcaacacaataagaaaagagaaggaggagaaccggatgagcaaaggagaagaacttttcactggagttgtcccaattcttgttgaattagatggtgatgttaatgggcacaaattttctgtccgtggagagggtgaaggtgatgctacaaacggaaaactcacccttaaatttatttgcactactggaaaactacctgttccgtggccaacacttgtcactactctgacctatggtgttcaatgcttttcccgttatccggatcacatgaaacgtcatgactttttcaagagtgccatgcctgaaggttatgtacaggaacgcactatatctttcaaagatgacgggacctacaagacgcgtgctgaagtcaagtttgaaggtgatacccttgttaatcgtatcgagttaaagggtattgattttaaagaagatggaaacattcttggacacaaactcgagtacaactttaactcacacaatgtatacatcacggcagacaaacaaaagaatggaatcaaagctaacttcaaaattcgccacaacgttgaagatggttccgttcaactagcagaccattatcaacaaaatactccaattggcgatggccctgtccttttaccagacaaccattacctgtcgacacaatctgtcctttcgaaagatcctaacgaaaagcgtgaccacatggtccttcttgagtttgtaactgctgctgggattacacatggcatggatgagctctacaaaccgagctcgagcaaagcccgccgaaaggcgggcttttctgtgggcatgctgagcaagcttgcggccgcacccagcagcaggatgtagtcctgtaacccacca

SEQ ID NO: 18 - DNA, 910 bp, synthetic construct
  source 1..910: mol_type = other DNA
tgtagtcctgtaacccaccatctagagcggccgctaacaccgtgcgtgttgacaattttacctctggcggtgataatggttgcgatatcagctagcaataattttgtttaactttaagaaggagatataccatggtcagcaaaggagaagcggtgatcaaagagttcatgcgttttaaagtgcatatggagggaagcatgaatggccatgagtttgaaatcgagggggaaggggaaggacgcccatatgaaggcacacagacggcaaaattgaaggtgaccaaaggtgggccgctgcctttttcgtgggatattctgtctccccagtttatgtacggttctcgcgcattcatcaagcaccccgctgatattccagattactataagcaatcttttccagagggtttcaaatgggagcgtgttatgaacttcgaggatggcggagctgttacggttactcaggatacctcgctggaggatggtacgctgatctataaagtaaagttacgcggcacgaatttccctccagacggtcctgtcatgcagaaaaaaacgatgggttgggaagcgtcaacggagcgtttgtacccggaagatggcgtgttgaaaggtgatattaagatggctcttcgccttaaggatggggggcgctacttagctgatttcaaaaccacatacaaagcgaagaagccagtacagatgcctggtgcgtacaatgttgaccgcaaattggacatcacaagtcacaatgaggattacactgtggtcgagcagtatgagcgtagtgaaggtcgccactctacgggcggaatggatgagctgtacaagtaaccgagctcgagcaaagcccgccgaaaggcgggcttttctgtggggatatctctagacagaattctgctgagcgtacctgt

SEQ ID NO: 19 - DNA, 1818 bp, synthetic construct
  source 1..1818: mol_type = other DNA
gcgcgaagttatcctacgaattcgtcaaagctttaacaccgtgcgtgttgacaattttacctctggcggtgataatggttgcagctagcaataattttgtttaactttaagaaggagatataccatgagcaaaggagaagaacttttcactggagttgtcccaattcttgttgaattagatggtgatgttaatgggcacaaattttctgtccgtggagagggtgaaggtgatgctacaaacggaaaactcacccttaaatttatttgcactactggaaaactacctgttccgtggccaacacttgtcactactctgacctatggtgttcaatgcttttcccgttatccggatcacatgaaacgtcatgactttttcaagagtgccatgcctgaaggttatgtacaggaacgcactatatctttcaaagatgacgggacctacaagacgcgtgctgaagtcaagtttgaaggtgatacccttgttaatcgtatcgagttaaagggtattgattttaaagaagatggaaacattcttggacacaaactcgagtacaactttaactcacacaatgtatacatcacggcagacaaacaaaagaatggaatcaaagctaacttcaaaattcgccacaacgttgaagatggttccgttcaactagcagaccattatcaacaaaatactccaattggcgatggccctgtccttttaccagacaaccattacctgtcgacacaatctgtcctttcgaaagatcctaacgaaaagcgtgaccacatggtccttcttgagtttgtaactgctgctgggattacacatggcatggatgagctctacaaaccgagctcgagcaaagcccgccgaaaggcgggcttttctgtgggcatgctgagcaagcttgcggccgcacccagcagcaggatgtagtcctgtaacccaccatctagagcggccgctaacaccgtgcgtgttgacaattttacctctggcggtgataatggttgcagctagcaataattttgtttaactttaagaaggagatataccatggtcagcaaaggagaagcggtgatcaaagagttcatgcgttttaaagtgcatatggagggaagcatgaatggccatgagtttgaaatcgagggggaaggggaaggacgcccatatgaaggcacacagacggcaaaattgaaggtgaccaaaggtgggccgctgcctttttcgtgggatattctgtctccccagtttatgtacggttctcgcgcattcatcaagcaccccgctgatattccagattactataagcaatcttttccagagggtttcaaatgggagcgtgttatgaacttcgaggatggcggagctgttacggttactcaggatacctcgctggaggatggtacgctgatctataaagtaaagttacgcggcacgaatttccctccagacggtcctgtcatgcagaaaaaaacgatgggttgggaagcgtcaacggagcgtttgtacccggaagatggcgtgttgaaaggtgatattaagatggctcttcgccttaaggatggggggcgctacttagctgatttcaaaaccacatacaaagcgaagaagccagtacagatgcctggtgcgtacaatgttgaccgcaaattggacatcacaagtcacaatgaggattacactgtggtcgagcagtatgagcgtagtgaaggtcgccactctacgggcggaatggatgagctgtacaagtaaccgagctcgagcaaagcccgccgaaaggcgggcttttctgtgggtctagacagaattctgctgagcgtacctgt

SEQ ID NO: 20 - DNA, 1873 bp, synthetic construct
  source 1..1873: mol_type = other DNA
gcgcgaagttatcctacgaattcgtcaaagctttaacaccgtgcgtgttgacaattttacctctggcggtgataatggttgcgatatcagctagcaataattttgtttaactttaagaaggagatataccatgcttgatctgaaattgggaggcttggctgctgcttgtttgctggtctgttctacggcacttgccgcacctttaccagacacccctggggcgccttttccagcagttgctaacttcgatcgttctggaccttacgctactagcaatcaatccgaaggtccctcatgtcgcatctaccgtccctctaatttgggacaggggggtgtgcgtcacccagtaattttgtggggaaacggtacgggcacaggtccctccacctatgcgggccttttatcccattgggcctcgcatggcttcgtcgtagcggcggctgagacgtcgaatgccggtacagggcgcgaaatgctggcctgtttggactatcttgtgcgcgaaaatgataatccctatgggacttatgctggtaagttgaacacaggacgtgtggggacatccggtcactcccaaggcggcggcggttcaattatggcggggcaggatacccgtgtgcgtacgacggcaccaattcaaccttacaccattggactgggccatgatagcgcctcacaacgtcgccaacaggggccaatgttccttatgtccggtggcggcgacactatcgctatcccctatcttaacgcgcagccagtgtatcttcgcgccaatgtacccgttttctggggcgagcgccgttacgtttctcatttcgagcccgtgggagacggtggtgcatatcgtggccccagcacggcctggtttcgtttccagttgatggacgatcagtcagcgcgtggtactttttatgggactctgtgctccctgtgttcctcattattgtggagtgtcgagcgccgtggcttctagagatgaatacgtataccaggtgtcctgtgagcagcgaaagcctaaacgggtctagagattaaagaggagaaatactagatgagcaaaggagaagaacttttcactggagttgtcccaattcttgttgaattagatggtgatgttaatgggcacaaattttctgtccgtggagagggtgaaggtgatgctacaaacggaaaactcacccttaaatttatttgcactactggaaaactacctgttccgtggccaacacttgtcactactctgacctatggtgttcaatgcttttcccgttatccggatcacatgaaacgtcatgactttttcaagagtgccatgcctgaaggttatgtacaggaacgcactatatctttcaaagatgacgggacctacaagacgcgtgctgaagtcaagtttgaaggtgatacccttgttaatcgtatcgagttaaagggtattgattttaaagaagatggaaacattcttggacacaaactcgagtacaactttaactcacacaatgtatacatcacggcagacaaacaaaagaatggaatcaaagctaacttcaaaattcgccacaacgttgaagatggttccgttcaactagcagaccattatcaacaaaatactccaattggcgatggccctgtccttttaccagacaaccattacctgtcgacacaatctgtcctttcgaaagatcctaacgaaaagcgtgaccacatggtccttcttgagtttgtaactgctgctgggattacacatggcatggatgagctctacaaaccgagctcgagcaaagcccgccgaaaggcgggcttttctgtgggcatgctgagcgatatcaagcttgcggccgcacccagcagcaggatgtagtcctgtaacccacca

SEQ ID NO: 21 - DNA, 1975 bp, synthetic construct
  source 1..1975: mol_type = other DNA
gcgcgaagttatcctacgaattcgtcaaagctttaacaccgtgcgtgttgacaattttacctctggcggtgataatggttgcgatatcagctagcaataattttgtttaactttaagaaggagatataccatgaagaagaacaaaacccttttggcattgtgcctgggcacgggtttattagcatcgggtcaaacacaggctttctggttcggatcgagcggctatacgcagactaagtacccgatcgtgttgggccacggtatgcttggattcgattcgattctgggggtagactattggtacggaattccggccgccttgcgccgtgacggcgcttctgtctatgtaaccgaggtctctcaattggacacaagcgaggctcgtggcgaacaattattacagcaagtcgaagacattgtagccatctcaggcaaaggcaaagtcaacttaatcggccacagtcacgggggaccaaccactcgctacgtggccgcggtacgcccagacttggttgcgtcagtaacgtctgttggagccccgcataaaggatccgcaacggccgactttcttaaagggatcagtgaaggtccagctggcccagtcgctactccagtattggtaggcattgtaaatgggctgggcgcactgattaacttcctttcaggatcgagctcaacgacgccccaaaacgcgttaggatccttagaatctcttaattccgaaggtgcagcacgctttaatgctaagtttccgcagggcattcctacctctgcatgcggagaaggcgcatattctgtgaacggcgtccgttattactcatggtcagggacaagtcccctgacgaacgtactggacccctcggacttgttattaggcgcatcgagtttaacattcgggagcgaggcaaacgatggcttagtaggtcgttgcagcagccgtatgggccaagtgattcgcgacaactaccgtatgaaccatcttgatgaagtgaaccagacattgggtcttacgagtctgttcgagacagacccggtgacggtttaccgccaacatgcaaaccgtcttaaaaacgctggcctgtagagatgaatacgtataccaggtgtcctgtgagcagcgaaagctctagagattaaagaggagaaatactagatgagcaaaggagaagaacttttcactggagttgtcccaattcttgttgaattagatggtgatgttaatgggcacaaattttctgtccgtggagagggtgaaggtgatgctacaaacggaaaactcacccttaaatttatttgcactactggaaaactacctgttccgtggccaacacttgtcactactctgacctatggtgttcaatgcttttcccgttatccggatcacatgaaacgtcatgactttttcaagagtgccatgcctgaaggttatgtacaggaacgcactatatctttcaaagatgacgggacctacaagacgcgtgctgaagtcaagtttgaaggtgatacccttgttaatcgtatcgagttaaagggtattgattttaaagaagatggaaacattcttggacacaaactcgagtacaactttaactcacacaatgtatacatcacggcagacaaacaaaagaatggaatcaaagctaacttcaaaattcgccacaacgttgaagatggttccgttcaactagcagaccattatcaacaaaatactccaattggcgatggccctgtccttttaccagacaaccattacctgtcgacacaatctgtcctttcgaaagatcctaacgaaaagcgtgaccacatggtccttcttgagtttgtaactgctgctgggattacacatggcatggatgagctctacaaaccgagctcgagcaaagcccgccgaaaggcgggcttttctgtgggcatgctgagcgatatcaagcttgcggccgcacccagcagcaggatgtagtcctgtaacccacca

SEQ ID NO: 22 - DNA, 1056 bp, synthetic construct
  source 1..1056: mol_type = other DNA
gcgcgaagttatcctacgaattcgtcaaagctttaacaccgtgcgtgttgacaattttacctctggcggtgataatggttgcgatatcattatattaaataaaatattttaacatttcggttgagcaaatttatatttgttttcaagagctttgaatttctggggtgggaaattccttaaaagaggaatatcctaccttttttgcgtacaaaatattatttggaggtgtgatttatgagcaaaggagaagaacttttcactggagttgtcccaattcttgttgaattagatggtgatgttaatgggcacaaattttctgtccgtggagagggtgaaggtgatgctacaaacggaaaactcacccttaaatttatttgcactactggaaaactacctgttccgtggccaacacttgtcactactctgacctatggtgttcaatgcttttcccgttatccggatcacatgaaacgtcatgactttttcaagagtgccatgcctgaaggttatgtacaggaacgcactatatctttcaaagatgacgggacctacaagacgcgtgctgaagtcaagtttgaaggtgatacccttgttaatcgtatcgagttaaagggtattgattttaaagaagatggaaacattcttggacacaaactcgagtacaactttaactcacacaatgtatacatcacggcagacaaacaaaagaatggaatcaaagctaacttcaaaattcgccacaacgttgaagatggttccgttcaactagcagaccattatcaacaaaatactccaattggcgatggccctgtccttttaccagacaaccattacctgtcgacacaatctgtcctttcgaaagatcctaacgaaaagcgtgaccacatggtccttcttgagtttgtaactgctgctgggattacacatggcatggatgagctctacaaaccgagctcgagcaaagcccgccgaaaggcgggcttttctgtgggcatgctgagcgatatcaagcttgcggccgcacccagcagcaggatgtagtcctgtaacccacca

(19) United States
(12) Patent Application Publication
(10) Pub. No.: US 2026/0209478 A1
(43) Pub. Date: Jul. 23, 2026

(54) AUTODEGRADABLE PLASTICS

(71) Applicant: E.V.A BIOSYSTEMS LIMITED, EDINBURGH (GB)

(72) Inventor: Alexander Joseph SPEAKMAN, Edinburgh (GB)

(73) Assignee: E.V.A BIOSYSTEMS LIMITED, EDINBURGH (GB)

(21) Appl. No.: 19/141,246

(22) PCT Filed: Dec. 21, 2023
(86) PCT No.: PCT/EP2023/087436

§ 371 Date: Jun. 19, 2025

(30) Foreign Application Priority Data
Dec. 22, 2022 (GB) 2219601.8

(51) Int. Cl. C08K 11/00; C12N 11/096; C12N 15/63

(52) U.S. Cl. CPC C08K 11/00; C12N 11/096; C12N 15/635; C08K 2201/018

(57) ABSTRACT

Disclosed is an autodegradable plastic material impregnated with a genetically engineered bacteria, in which the bacteria are engineered to include a genetic switch that is activated by an environmental factor. Activation of the switch causes expression of an enzyme that degrades the plastic.

### FIELD OF THE INVENTION

The present invention relates to polymer materials derived from the petrochemical industry and/or bio-sourced, impregnated with bacteria that degrade the polymeric material on stimulation by a specific environmental trigger. The present invention further relates to bacteria for impregnating a polymer material, which bacteria, on stimulation by a specific environmental trigger, affect a characteristic, such as the integrity, of the plastic material.

### BACKGROUND OF THE INVENTION

Plastic-eating bacteria have been being developed for a number of years in an attempt to reduce plastic pollution. For example, Ideonella sakaiensis is capable of breaking down and consuming the plastic polyethylene terephthalate (PET), using it as both a carbon and energy source. While this technology could lead to a more sustainable future, it is not the silver bullet for the global plastic problem. There are already billions of tons of plastic waste in landfills and in the ocean, the sheer volume of which cannot be solved by bioremediation alone. As a result, there is a need to develop polymeric materials that are biodegradable as well as means to degrade such materials.

US 2014/0303278 describes a polymer/biological entities ‘alloy’ in which a polymer is mixed with biological entities, including enzymes and microorganisms that degrade the polymer, during a heat treatment. The heat treatment is performed at a temperature above room temperature and the biological entities are resistant to the temperature. The ‘alloy’ undergoes total degradation under aqueous conditions (e.g. in crystalline mineral water) while remaining stable under dry conditions. Degradation may occur at an acceptable rate under temperature, pH and humidity conditions that are compatible with those generally encountered in the natural environment. Interestingly, the microorganisms are present and kept in the alloy in a dehydrated state until biodegradation is sought. In addition, the microorganisms are naturally pre-disposed to degrade the polymer (i.e. they are not genetically modified).

An alternative to dehydration is to include bacteria in a sporulated form, as suggested in U.S. Pat. No. 9,974,311, for the inclusion of bacteria that degrade organic material into a mouldable thermoplastic, thereby controlling odours arising from organic matter.

JP 2013/209587 describes biodegradable plastic products with regulated degradation rates in which the biodegradable plastic product comprises a polyhydroxyalkanoic acid (‘PHA’) component in which a strain of bacteria producing polymerase and/or spores of the strain have been immobilised. The spores are of a micro-organism (preferably bacillus) that produces a polyhydroxyalkanoic acid degrading enzyme. Alternatively, the bacterium is a thermophilic bacterium that produces a polyhydroxyalkanoic acid degrading enzyme. The bacterial spores are added to the plastic at the time of moulding or added to the plastic surface before the plastic has solidified. Apparently, the rate of degradation of the biodegradable plastic products can be regulated by adjusting the number of viable microorganisms or viable spores. When anchored inside the product, after the surface of the product has undergone biodegradation, the micro-organisms and/or spores present inside the product accelerate degradation by multiplying.

WO 2022/026137 discloses an auto-biodegradable absorbent article (e.g. nappy or sanitary towels) formed, at least in part, from a biodegradable polymer that includes an inactivated microorganism product. The absorbent article contains one or more microorganisms that are designed to secrete an enzyme that degrades the biopolymer. The microorganism naturally secretes the enzyme or can be genetically modified to secrete the enzyme. The microorganisms or bacteria incorporated into the absorbent article are particularly selected based upon their salt tolerance and enzyme production at certain salt concentrations. The inactivated microorganism product is configured to activate upon contact with a salt-containing liquid that has a salt concentration of about 50 millimolar or greater.

Accordingly, it is against this background that the present invention has been devised. In particular, there is a need to harness the ability of bacteria to degrade polymeric materials, including plastic, in which the bacteria are activated at precise environmental trigger points. This allows the material to have a suitable life span even when stored in, or storing, aqueous solutions.

### SUMMARY OF THE INVENTION

In accordance with a first aspect of the invention, there is provided a plastic material impregnated with a genetically engineered bacteria, in which the bacteria are engineered to include a genetic switch that is activated by an environmental factor, and wherein activation of the switch causes the bacteria to affect a characteristic, such as the integrity, of the plastic material or causes a characteristic of the bacteria to change.

In a second aspect of the invention, there is provided a method of manufacturing an autodegradable polymer material or plastic of the first aspect, wherein a feedstock or powdered form of the material may be coated or mixed with recombinant biological entity.

In a third aspect of the invention, there is provided a genetically modified (recombinant) bacteria for use in the first and/or second aspects of invention. Preferably, the genetically modified (recombinant) bacteria degrade polymeric materials and plastic, wherein the bacteria are engineered to express a genetic switch that is activated by an environmental factor and an enzyme that degrades the plastic, and wherein expression of the enzyme is activated by the genetic switch.

### DETAILED DESCRIPTION OF THE INVENTION

Accordingly, the first aspect of the present invention encompasses a plastic material impregnated with a genetically engineered bacteria, in which the bacteria are engineered to include a genetic switch that is activated by an environmental factor, and wherein activation of the switch causes the bacteria to affect a characteristic, such as the integrity, of the plastic material or causes a characteristic of the bacteria to change. Preferably, activation of the switch causes, enhances or increases expression of an enzyme that affects a characteristic of the plastic material. More preferably, activation of the switch causes, enhances or increases expression of an enzyme that degrades the plastic material. In this way, the plastic-embedded bacteria are pre-disposed (e.g. genetically modified) to biodegrade the plastic when exposed to an environmental trigger.

As such, in a preferred embodiment of the first aspect, there is disclosed a plastic material impregnated with a genetically engineered bacteria, in which the bacteria are engineered to include a genetic switch that is activated by an environmental factor, and wherein activation of the switch causes, enhances or increases expression of an enzyme that degrades the plastic material.

Expressed in another way, the invention resides in a polymer material or plastic that incorporates genetically engineered bacteria that respond to one or more environmental stimuli, e.g. to break down the material in which they are encased. In this way the invention provides a plastic/polymer material that self-degrades only under specific and selected environmental conditions. With the incorporation of specific genetically modified bacteria, the material does not contribute to plastic waste and does not compromise on the qualities that make plastic such a useful material.

The term plastic is used to encompass polymeric material that is derived from the petrochemical industry and/or bio-sourced, the latter being bio-based, biodegradable and/or compostable plastics. For the avoidance of doubt, bio-based plastics are fully or partially made from biological resources, rather than fossil raw materials. They are not necessarily compostable or biodegradable. Biodegradable and compostable plastics biodegrade in certain conditions and may be made from fossil-fuel based materials.

In one embodiment, the switch may be temporarily (transiently) activatable or permanently activatable by an environmental factor. A temporary or transient switch is one in which enzyme expression is activated only when and while the polymeric material is subjected to the environmental factor. For example, when a material of the invention is in seawater, enzyme expression will stop if and when the material is taken out of the seawater. Such a situation is useful if, for example, a splash of salt water hits a plastic bottle that is still in use (e.g. during cooking). That splash of salt water will not cause a hole in the bottle over time. In the alternative, a permanent switch will activate enzyme expression once the plastic material has been subjected to the environmental factor, and enzyme expression will continue even if the environmental factor is removed.

In an embodiment, the switch may be activatable only while exposed to the environmental factor. Expressed in another way, if the specific environmental factor is not present, even if the specific environmental factor was encountered previously, there is no degradation (or no continued degradation) by the bacteria. It will be appreciated that this situation only arises when the switch is transiently activatable, as described herein above.

In a further embodiment, the switch may activate once exposed to the environmental factor. Expressed in another way, if the environmental factor has been encountered, degradation by the bacteria continues even if the environmental factor is removed.

In a particular embodiment, the environmental factor may be a change in pH, light, temperature, an electric current, microenvironment, or in the presence of ions or ligands. As an example, the environmental factor may be a change or a drop in pH, an example of which is a low pH environment such as that found in a landfill setting. In such an example, the bacteria may be engineered to include a pH-activated promoter, such as ADAR (arginine-dependent acid resistance) or GDAR (glutamate-dependent acid-resistance) promoter systems.

In another example, the environmental factor may be the presence of sodium and/or chloride (e.g. sodium chloride, or sodium ions and/or chloride ions), an increase in salinity, a change in osmotic potential, and/or a high salinity environment (around 33 g/L or above) such as that found in sea water. In this example, the bacteria may be engineered to include a salt-activated promoter such as OtsB, an osmotic promoter system such as OsmY, or a Na⁺-sensitive or salt-dependent riboswitch.

For the avoidance of doubt, a riboswitch is a regulatory segment of a mRNA molecule that binds a small molecule, resulting in a change in production of proteins encoded by the mRNA. Thus, a mRNA that contains a riboswitch is directly involved in regulating its own activity, in response to the concentrations of its effector molecule. An example of a riboswitch class, previously called the ‘DUF1646 motif’, whose members selectively sense Na⁺ and regulate the expression of genes relevant to sodium biology is described by White N., et al ((2022) Nature Chemical Biology, 18, 878-885).

It will be appreciated that the switches may have sequences found in nature, or may be engineered and/or mutated to enhance function and/or tuned to a particular environment or desired result or outcome.

In an embodiment, the bacteria may be capable of activation from, and optionally to, a dormant state. Expressed in another way, the bacteria may be present in the plastic in a dormant state. In this way, the bacteria conserve energy and resources until the plastic degrading function is required. It also enables the bacteria to remain intact and viable during any processing of the plastic. It will be appreciated that the plastic may be impregnated with bacteria that are already in a dormant state, or the bacteria may be impregnated in an active state and switched or converted to a dormant state once the bacteria are embedded in the plastic material. Switching or converting to a dormant state from an active state or switching or converting to an active state from a dormant state is an example of a change in a characteristic of the bacteria.

A dormant state may be where the bacteria are in the form of (endo-) spores. The spores of spore-forming bacteria are dormant bodies that carry all the genetic material, and some biological machinery such as ribosomes and some enzymes, that is found in the vegetative form, but do not have an active metabolism. They are much more resistant against heat, dryness, and other negative ambient conditions than the vegetative form, so they act as a mean of survival during hard times. When the environmental conditions turn favourable, spores germinate to vegetative cells. Spore-forming bacteria are Firmicutes and are psychrotrophic, mesophilic or thermophilic, aerobic or anaerobic, and use minerals or organic molecules for energy formation. Examples of spore-forming bacteria include Bacillus (aerobic) and Clostridium (anaerobic) species.

Non-spore forming bacteria, such as Enterobacter, Klebsiella, Escherichia coli and Vibrio natrigens, lose their culturability when subjected to stress, leading to a transient dormancy state.

In another embodiment, the plastic material may be in the form of feedstock. Preferably, the plastic material may be in the form of filaments for use in 3D printing, beads or pellets for use in injection moulding, or sheets for use in vacuum moulding. Feedstock is unprocessed material, a primary commodity, or a basic material that is used to produce goods, finished goods, or intermediate materials that are feedstock for future finished products. In this way, the bioplastic material of the invention may be processed and used using conventional and existing manufacturing equipment to create plastic products while adding the benefit of environmental degradation.

In a particular embodiment, the plastic material may be a thermoplastic, such as a polythene, high-density polyethene (HDPE), low-density polyethene (LDPE), polycarbonates, acrylics, polyamides, polystyrenes, polypropylenes, acrylonitrile butadiene styrenes (ABS), polyesters. Polycaprolactone (PCL) and polyethylene terephthalate (PET) are particular examples. Such plastics tend to melt at a low enough temperature to enable the bacteria to retain their viability and not to pasteurise non-sporulated bacteria. The plastic material may be processed for use as packaging and/or single-use plastics.

It will be appreciated that the enzyme expressed by the bacteria when the switch is activated is specific to the plastic material in which the bacteria are embedded and is selected with this in mind when putting the present invention into effect. In a particular example, the enzyme might be an esterase, such as a PETase, or a derivative or mutation thereof. Examples of suitable enzymes also include, Pseudomonas hydrolase which degrades LDPE (Tribedi P. and Sil A. K. (2013) Environmental Science and Pollution Research, 20, 4146-4153), MHETases which also degrade PET plastics (Jerves C. et al (2021) ACS Catal., 11 (18), 11626-11638), Proteinase K which degrade polylactic acid (PLA) (Huang Q. et al (2020) Biomacromolecules, 21 (8), 3301-3307), lipases from various sources which can degrade PCL (Gan Z. et al (1997) Polymer Degradation and Stablity, 56 (2), 209-213), and hydrolase and PHB depolymerase which can degrade polyhdroxybutyrate (PHB) (Altaeee N. et al (2016) SpringerPlus, 5, 762). It will be appreciated that the enzyme expressed may have a naturally occurring or engineered sequence or be the whole or part of a natural or engineered sequence.

In a yet further embodiment, activation of the genetic switch may switch the bacteria from an active to a dormant state (and/or vice versa) or the bacteria may further include one or more additional genetic switches that switch the bacteria from an active to a dormant state (and/or vice versa).

In a particular embodiment, the bacteria may be present in the plastic in a dormant state and further include one or more additional genetic switches that activate the bacteria from the dormant state. In this way, the bacteria do not have to be thermostable or able to withstand processing conditions for plastics manufacture that would otherwise render the bacteria non-viable. The bacteria are also able to conserve energy and resources until the resources are required to degrade the plastic. In a particular example, the dormant state of the bacteria may be as a spore.

In a particular embodiment the additional genetic switch may be temperature sensitive. More specifically, the additional genetic switch may be, or may be derived from, a thermo-sensitive promoter, such as a heat shock promoter or promoter system (Roncarati D. and Scarlato V. (2017) FEMS Microbiology Reviews, 41 (4), 549-574), a thermosensitive RNA thermoswitch (Johansson J. et al (2002) Cell, 110 (5), 551-561) and/or heat sensitive G-quadruplex DNA nanostructures (Wieland M. and Hartig J. (2007) Chemistry and Biology, 14 (7), 757-763).

In addition to the environmental control, heat-controlled switches may be used throughout the manufacturing process as a means of controlling the bacteria, and/or expression or activity of the plastic-degrading enzyme(s) within the bacteria, at different stages of manufacture. As an example, the genetic switch may harness heat-induced recombinase expression to induce permanent genetic changes (Meinke G. et al (2016) Chem. Rev., 116 (20), 12785-12820) in response to transient heat pulses provided to the bacteria from a heated extruder. By expanding the recombinase system, a bacteria may be made to “count” how many times it has been heated (once=in feedstock, twice=manufactured into a part) and change its gene expression accordingly.

The engineered genetic circuitry may also be used as a means of deactivating bacteria while in feedstock and reactivating after manufacture, to improve bacterial resistance to the high temperatures of extrusion (such as by forming spores), only producing temperature or other environmental factor sensitive products after the final exposure to heat, or to avoid wasting bacterial energy and resources by producing desired products before they are needed.

Also encompassed by the present invention is a method of manufacturing an autodegradable (e.g. thermoplastic) polymer material or plastic as described herein, wherein a feedstock or powdered form of the material may be coated or mixed with recombinant biological entity. When a powdered form of the material is used, the resulting powdered mixture may be formed into feedstock. Optionally the feedstock may be heated and formed. In this way, the biological entity may be uniformly distributed throughout the final product.

In a particular embodiment, the feedstock may be heated to around 70-80° C. and may be formed into an extruded filament, beads, sheets or a final product. It will be understood that extruded filaments are for use in 3D printing, beads are for use in injection moulding and sheets are used for vacuum moulding.

In an embodiment, the biological entity may be one or more proteins (e.g. enzymes), or bacteria as described herein.

Where the biological entity is bacteria, such as described herein, the bacteria may be mixed or coated in the form of spores or dormant bacteria. Alternatively, the bacteria may be mixed or coated in a vegetative state. Optionally, the bacteria may be converted to a dormant state or spores when mixed or coated. In one embodiment and as described hereinabove, the bacteria may be converted by the application of heat.

In a further embodiment, the bacteria may be re-activated from a dormant state or spore by the application of heat, or an environmental stimulus as described herein. It will be appreciated that, where two applications of heat are used, the heat may or may not be provided at the same temperature, intensity and/or from the same source.

In a yet further embodiment, the bacteria may be engineered to include a further, second genetic switch that is activated by the same or a different environmental factor(s) to the first genetic switch such that activation of one of the genetic switches causes a change in behaviour (e.g. causes, enhances or increases expression of an enzyme) or characteristics (e.g. changing from an active to a dormant state, or vice versa) of the bacteria, and activation of the other genetic switch changes or affects the characteristics, such as the integrity, of the plastic material. In a particular embodiment, activation of at least one of the first and second genetic switches may cause the bacteria to express an enzyme that degrades the plastic. Alternatively or in addition, at least one of the first and second genetic switches is a permanent switch. In a particular example, both the first and the second genetic switches may be permanent switches. Preferably, the activation of the first genetic switch precedes activation of the second genetic switch. In a particular embodiment, the first genetic switch is a permanent genetic switch, and the second genetic switch is activatable only after activation of the first genetic switch.

In another aspect, the present invention resides in genetically modified (recombinant) bacteria that degrade polymeric materials and plastic, wherein the bacteria are engineered to express a genetic switch that is activated by an environmental factor and an enzyme that degrades the plastic, and wherein expression of the enzyme is activated by the genetic switch. The bacteria, genetic switch, environmental factor and enzyme are all as described herein.

The present invention will now be described with reference to the following non-limiting examples and figures, in which:

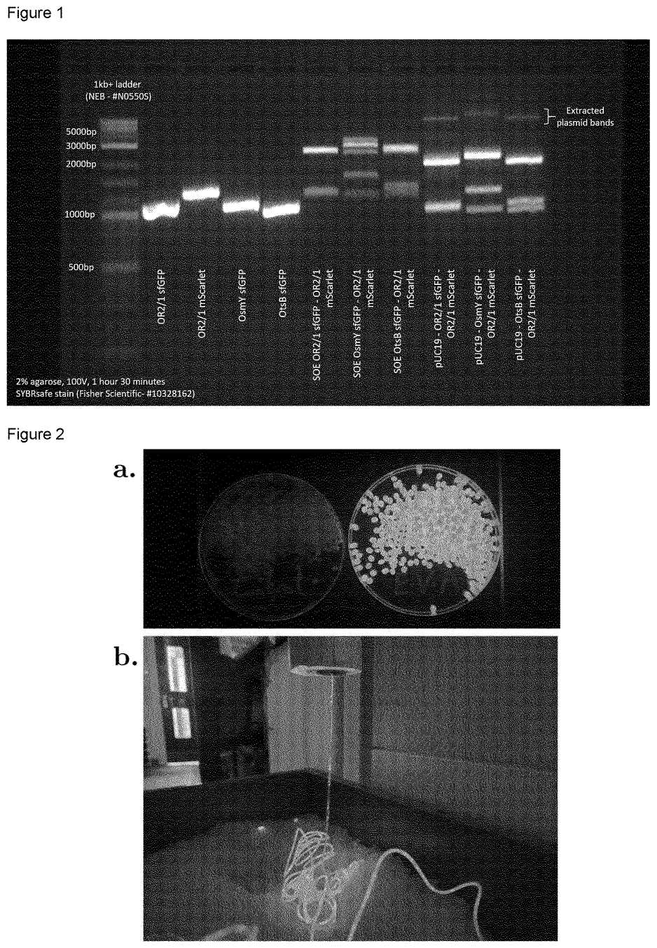

FIG. 1: Electrophoresis gel of DNA inserts encoding fluorescent protein products controlled by constitutively active (OR2/1) or NaCl induced (OsmY and OtsB) promoters. The gel shows each protein coding DNA section after amplification by PCR, followed by joining of each separate superfolder green fluorescent protein (sfGFP) product to the constitutively mScarlet product via splicing by overlap extension (SOE). Finally, joined DNA segments underwent restriction digest using EcoRI and were ligated into a digested pUC19 plasmid to be used for heat shock transformation of E. coli.

FIG. 2: Biomaterial feedstock prototypes. FIG. 2a) Raw plastic beads treated with E. coli bacteria engineered to express sfGFP. FIG. 2b) Extrusion of bacterial plastic beads into a 3D printer compatible filament, still expressing sfGFP. Both images are viewed through a blue light filter, with the subjects excited under blue light to cause the sfGFP to fluoresce.

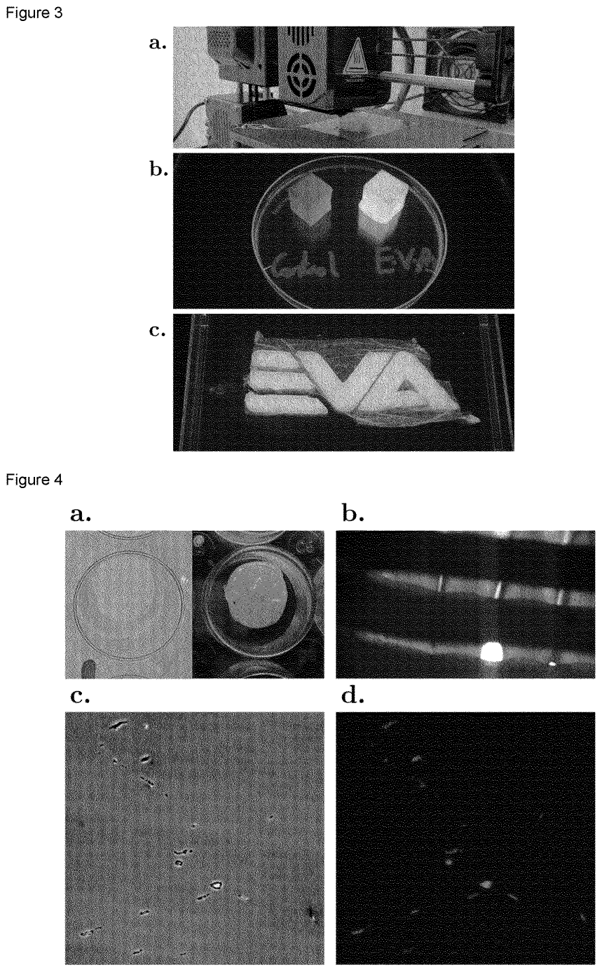

FIG. 3: Printing using smart biomaterial filaments. FIG. 3a) A test cube being printed using a 3D printer. FIG. 3b) Comparison between test cubes, both printed with filament manufactured as described herein, one with sfGFP expressing E. coli (EVA) and one without (Control). FIG. 3c) Print of “EVA” letters using a PCL/sfGFP expressing E. coli filament.

FIG. 4: Fluorescence microscopy of smart biomaterial printed parts. FIG. 4a) Standard photo of printed disc under normal light and fluorescence conditions. FIG. 4b) Low magnification bright field microscopy of printed disc. FIG. 4c) High magnification bright field microscopy of printed plastic disc. FIG. 4d) High magnification sfGFP fluorescence microscopy of printed disc.

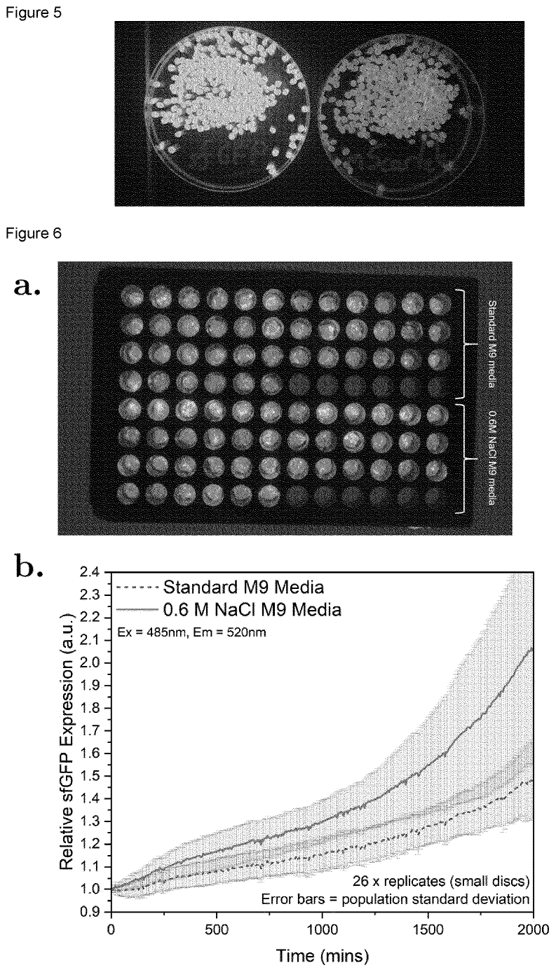

FIG. 5: Polycaprolactone beads treated with different engineered E. coli, one producing a green fluorescent protein (sfGFP), and the other producing a red fluorescent protein (mScarlet). Both use the same promoter for expression, only the protein has been changed.

FIG. 6: Salt-responsive biomaterial demonstration. An E. coli strain was engineered to produce sfGFP in a salt-dependent manner, then incorporated into plastic filament and printed into thin discs for scanning using a fluorescence plate reader. FIG. 6a) Printed discs in a 96-well microplate, top 4 rows contain standard M9 media, and the bottom 4 rows contain 0.6M NaCl supplemented M9 media (the same concentration of salt as seawater). FIG. 6b) Results of sfGFP fluorescence scanning over time.

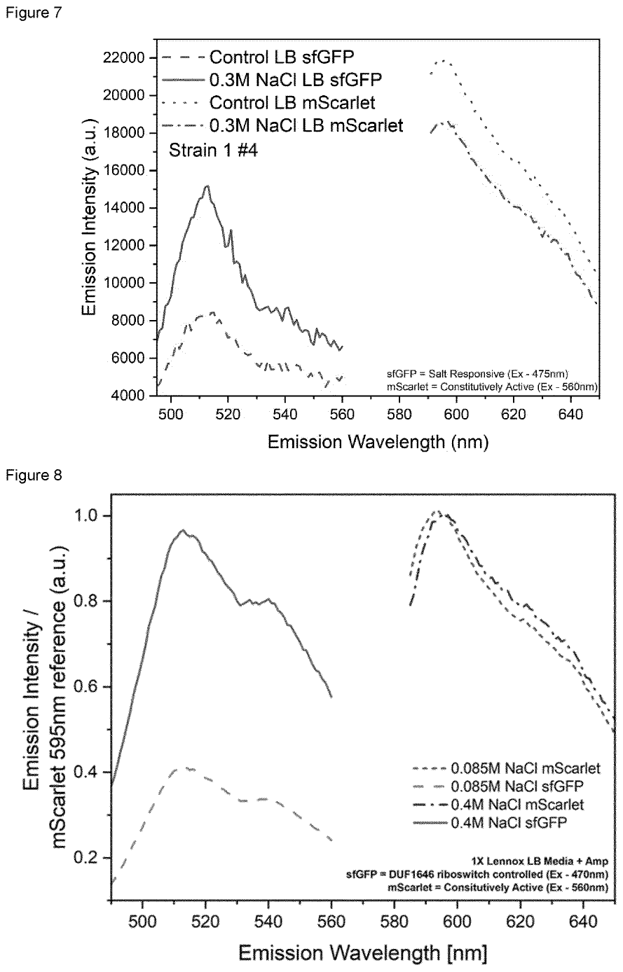

FIG. 7: Salt-responsive biomaterial demonstration. E. coli were engineered as in FIG. 6 but grown in LB media, instead of M9, and pelleted down and resuspended in PBS for scanning.

FIG. 8: Emission spectra of DUF1646-sfGFP E. coli in low salt concentration (0.085M) and high salt concentration (0.4M) media.

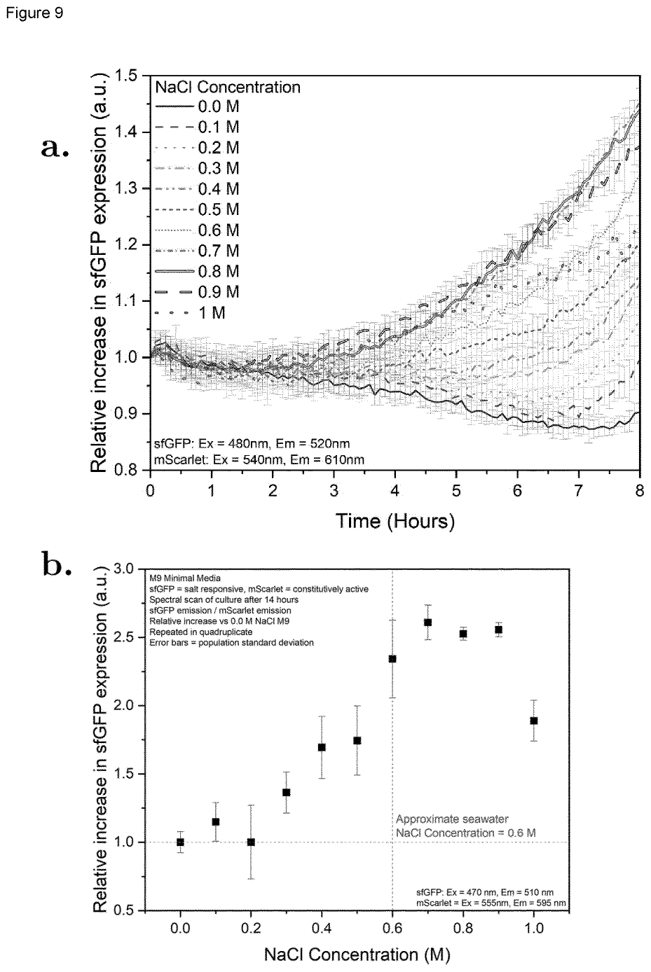

FIG. 9: Engineered salt-responsive bacteria in different NaCl concentrations. E. coli including sfGFP under the control of an OtsB promoter, and mScarlet under the constitutively active OR1/2 promoter. sfGFP intensity was referenced against mScarlet as a measure of cell density. FIG. 8a) 8-hour kinetics of sfGFP expression referenced against mScarlet. FIG. 8b) Relative sfGFP expression after 14 hours determined from peak emission wavelengths using full spectral scan.

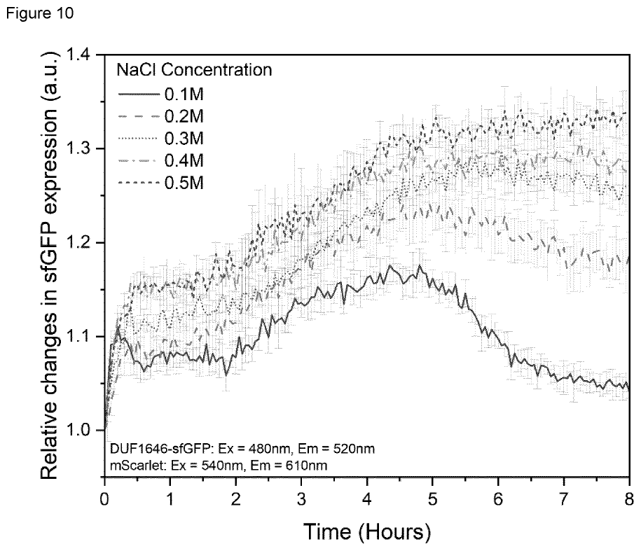

FIG. 10: 8-hour kinetics of DUF1646-sfGFP expression. sfGFP emission values were referenced against mScarlet emission values as a measure of cell density and normalised to initial start point to show the relative change over time. Three replicates were used to generate average and standard deviation values.

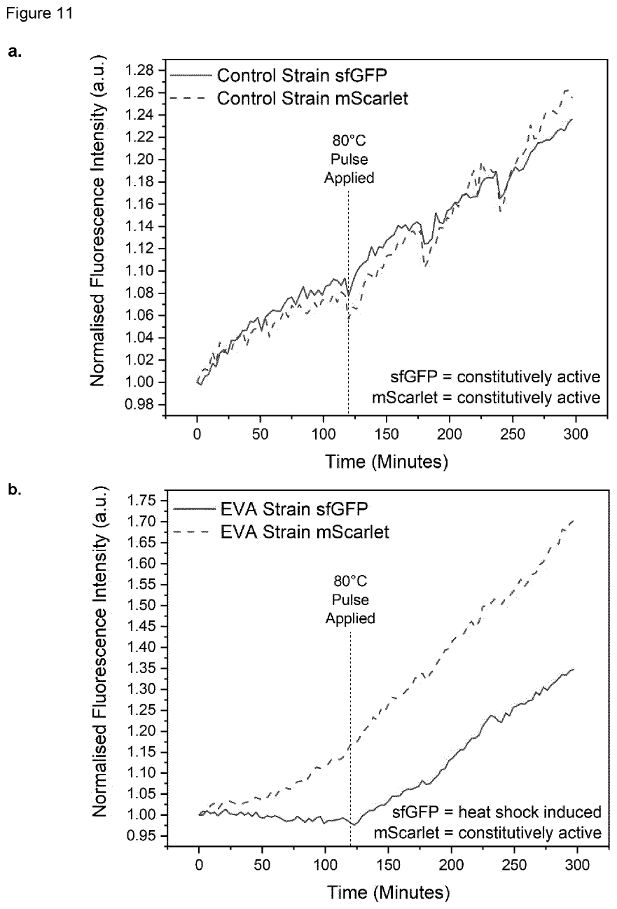

FIG. 11: Temperature induced elements of genetic circuits for use in smart biomaterials, demonstration of heat shock activation of engineered E. coli using HtrA promoter controlled sfGFP. FIG. 11a) 5-hour kinetics of sfGFP and mScarlet in a control strain, with an 80° C. heat shock after 2 hours. FIG. 11b) Kinetics of heat shock induced sfGFP strain.

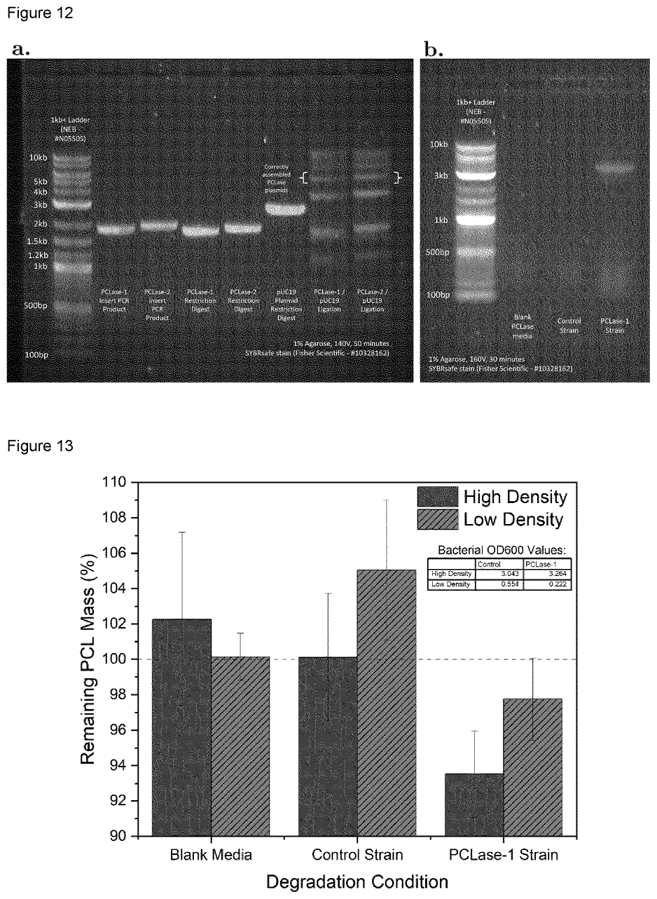

FIG. 12: Assembly and confirmation of PCLase encoding plasmid using PCR: FIG. 12a) Plasmids encoding PCLase-1 and PCLase-2 were each joined to a non-functional sfGFP using NEBuilder kit and amplified via PCR. PCR products and a pUC19 vector were then digested using HindIII and ligated together to produce a correctly assembled plasmid with a size of approximately 4.4 kb. FIG. 12b) Colony PCR of degradation experiment bacterial stocks was performed to confirm the presence of PCLase-1 within the strain being tested.

FIG. 13: Degradation of PCL by an engineered E. coli strain.

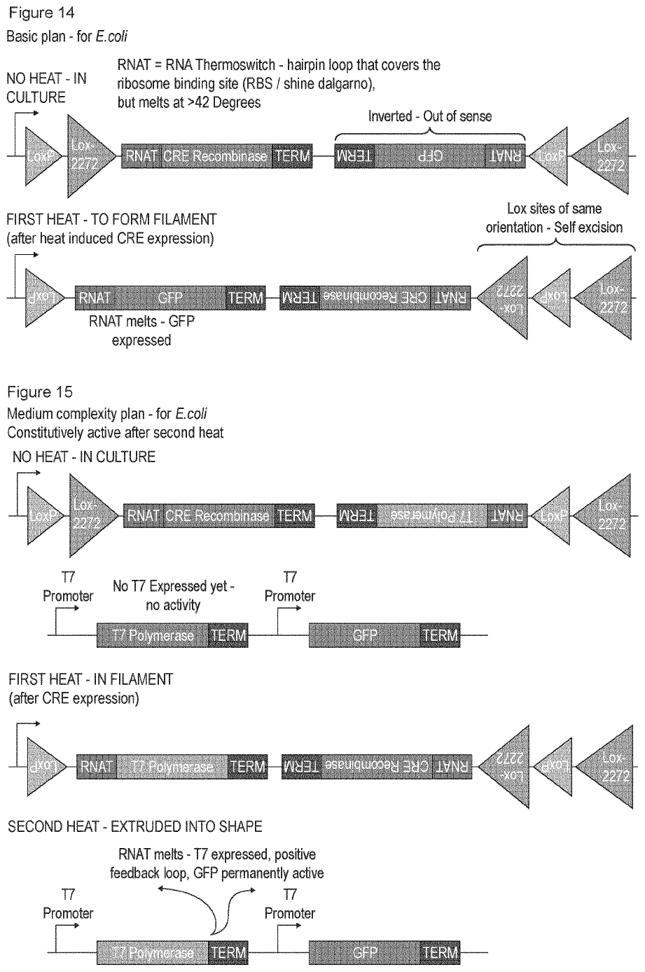

FIG. 14: Illustration of a simple heat shock control system in which E. coli are permanently induced to express GFP by a single extrusion heat shock. Triangle/arrows=recombination sites (point=orientation/directionality of recombination sites, based on Flex system (https://blog.addgene.org/plasmids-101-flex-vectors; U.S. Pat. No. 7,074,611)).

FIG. 15: Illustration of a medium complexity heat shock control system in which E. coli are permanently induced to express GFP after a second extrusion heat shock using a single recombinase system and a T7 polymerase feedback loop.

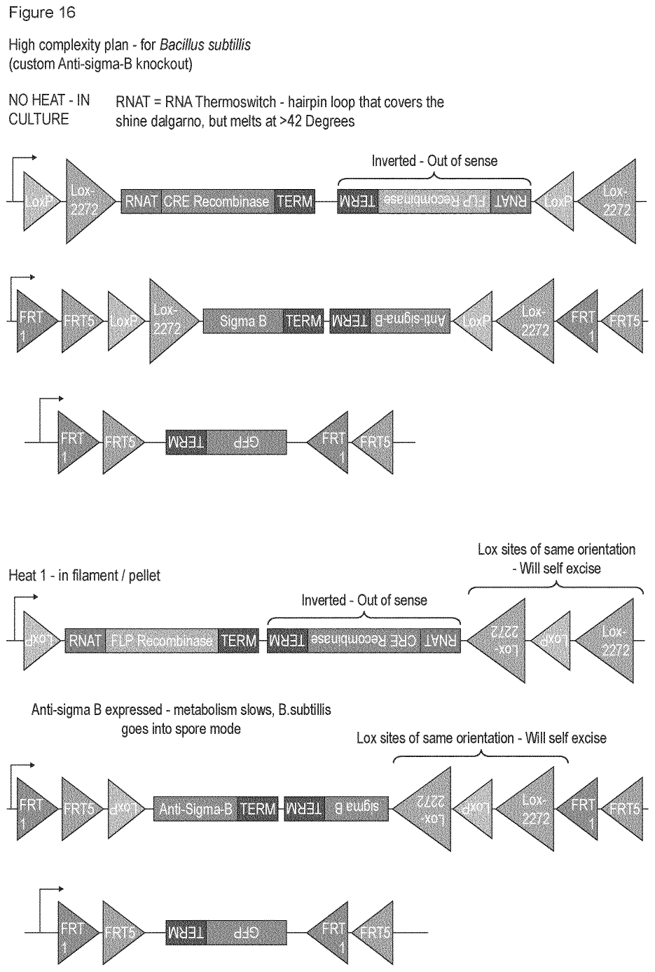

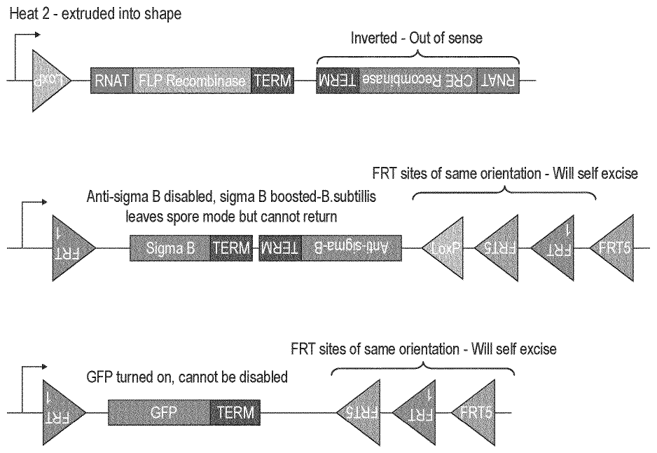

FIG. 16: Illustration of a high complexity heat shock control system in which B. subtilis spore formation is induced by first extrusion heating, deactivation of spores and permanent GFP expression is induced by a second extrusion heat shock using a dual recombinase system.

### METHODS

Engineering of E. coli

Plasmid insert sequences were designed using published articles, supplementary data, and GenBank submissions. Parts were organised as follows (5′-3′): Promoter/RBS—Product—Terminator. Restriction sites were included for insertion into a plasmid multiple cloning site (MCS) via sticky end ligation and for manipulation of DNA sequences if needed.

### Promoters and Ribosome Binding Sites (RBS):

OR2—OR1—Constitutively active (Shin J. and Noireaux V. (2010) J. Biolog. Eng., 4, 8). These were coupled to a T7 g10 phage ribosome binding site to enhance translation (Olins P.O. et al (1988) Gene, 73 (1), 227-235)
OtsB/OsmY—Osmotic pressure/salt induced (Rosenthal A. Z. et al (2006) Mol. Microbiol., 59 (3), 1052-1061). This sequence used the wild-type ribosome binding site that naturally occurs after OtsB promoter.
IbpA—heat shock promoter (Chuang S-E. et al (1993) Gene, 134 (1), 1-6)
DUF1646—Na⁺ sensitive riboswitch (White N. et al (2022) Nat. Chem. Biol.; 18 (8): 878-885).

### Products:

sfGFP—superfolder green fluorescent protein (Pédelacq J-D et al (2005) Nature Biotechnology, 24 (1), 79-8)
mScarlet—red fluorescent protein (Bindels Ds. et al (2016) Nature Methods, 14(1), 53-5)
PCLase—1-Hypothetical hydrolase (Li L. et al (2022) Front. Bioeng Biotechnol., 10)—100% sequence similarity to Genbank—WP_004373894.
PCLase-2—Triacylglycerol lipase—(Li L. et al supra) 100% sequence similarity to Genbank—WP_003239806

### Terminator:

T500 terminator (Greenblatt J. F. (2008) Cell, 132 (6), 917-918)

### Restriction Sites:

EcoRI (GAATTC)—Primary insertion restriction enzyme, 5′ of left/sfGFP and 3′ of right/mScarlet, used to insert combined gBlock products into plasmid.
HindIII (AAGCTT)—Flanking sfGFP gBlock. Used for inserting sfGFP only.
XbaI (TCTAGA)—Flanking mScarlet gBlock. Used for inserting mScarlet only.
NotI (GCGGCCGC)—3′ of left/sfGFP and 5′ of right/mScarlet.
EcoRV (GATATC)—Not present in all gBlocks, blunt end cut used to allow OR2—OR-1 promoters to be removed and to allow a new promoter sequence to replace it via primer extension.
Sequences were synthesised by IDT™ as gBlocks™ that combined a single promoter (e.g., OR2/1_sfGFP, OtsB_sfGFP, OsmY_sfGFP and OR2/1_mScarlet). Each part was flanked with primer sites for amplification by PCR, and various restriction sites for modular assembly (see sequences section).

Sequences

Left (sfGFP gBlock) Primers:
Forward-GCGCGAAGTTATCCTACGAAT (Sense) [SEQ ID NO: 1]
Reverse-TGGTGGGTTACAGGACTACA (AntiSense) [SEQ ID NO: 2]
Right (mScarlet gBlock) Primers:
Forward-GTAGTCCTGTAACCCACCATC (Sense) [SEQ ID NO: 3]
Reverse-ACAGGTACGCTCAGCAGAAT (AntiSense) [SEQ ID NO: 4]
PClase_FWD_16102023: GCGCGAAGTTATCCTACGAAT [SEQ ID NO: 5]
PClase_REV_16102023: CCCGTTTAGGCTTTCGCT [SEQ ID NO: 6]
sfGFP_FWD_16102023: GCAGCGAAAGCCTAAACG [SEQ ID NO: 7]
sfGFP_REV_16102023: TGGTGGGTTACAGGACTACA [SEQ ID NO: 8]
T7g10 leader sequence:
AGCTAGCAATAATTTTGTTTAACTTTAAGAAGGAGATATACC [SEQ ID NO: 9]
BBa_B0032 ribosome binding sequence:
TCTAGAGATTAAAGAGGAGAAATACTAG [SEQ ID NO: 10]
OR2-OR1 promoter:
TAACACCGTGCGTGTTGACAATTTTACCTCTGGCGGTGATAATGGTTGC [SEQ ID NO: 11]
T500 Terminator sequence:
CCGAGCTCGAGCAAAGCCCGCCGAAAGGCGGGCTTTTCTGT [SEQ ID NO: 12]
PCLase-1:
GCGCGAAGTTATCCTACGAATTCGTCAAAGCTTTAACACCGTGCGTGTTGACAATTTTACCT
CTGGCGGTGATAATGGTTGCGATATCAGCTAGCAATAATTTTGTTTAACTTTAAGAAGGAGA
TATACCATGCTTGATCTGAAATTGGGAGGCTTGGCTGCTGCTTGTTTGCTGGTCTGTTCTAC
GGCACTTGCCGCACCTTTACCAGACACCCCTGGGGCGCCTTTTCCAGCAGTTGCTAACTTCG
ATCGTTCTGGACCTTACGCTACTAGCAATCAATCCGAAGGTCCCTCATGTCGCATCTACCGT
CCCTCTAATTTGGGACAGGGGGGTGTGCGTCACCCAGTAATTTTGTGGGGAAACGGTACGGG
CACAGGTCCCTCCACCTATGCGGGCCTTTTATCCCATTGGGCCTCGCATGGCTTCGTCGTAG
CGGCGGCTGAGACGTCGAATGCCGGTACAGGGCGCGAAATGCTGGCCTGTTTGGACTATCTT
GTGCGCGAAAATGATAATCCCTATGGGACTTATGCTGGTAAGTTGAACACAGGACGTGTGGG
GACATCCGGTCACTCCCAAGGCGGCGGCGGTTCAATTATGGCGGGGCAGGATACCCGTGTGC
GTACGACGGCACCAATTCAACCTTACACCATTGGACTGGGCCATGATAGCGCCTCACAACGT
CGCCAACAGGGGCCAATGTTCCTTATGTCCGGTGGCGGCGACACTATCGCTATCCCCTATCT
TAACGCGCAGCCAGTGTATCTTCGCGCCAATGTACCCGTTTTCTGGGGCGAGCGCCGTTACG
TTTCTCATTTCGAGCCCGTGGGAGACGGTGGTGCATATCGTGGCCCCAGCACGGCCTGGTTT
CGTTTCCAGTTGATGGACGATCAGTCAGCGCGTGGTACTTTTTATGGGACTCTGTGCTCCCT
GTGTTCCTCATTATTGTGGAGTGTCGAGCGCCGTGGCTTCTAGAGATGAATACGTATACCAG
GTGTCCTGTGAGCAGCGAAAGCCTAAACGGG [SEQ ID NO: 13]
PCLase-2:
GCGCGAAGTTATCCTACGAATTCGTCAAAGCTTTAACACCGTGCGTGTTGACAATTTTACCT
CTGGCGGTGATAATGGTTGCGATATCAGCTAGCAATAATTTTGTTTAACTTTAAGAAGGAGA
TATACCATGAAGAAGAACAAAACCCTTTTGGCATTGTGCCTGGGCACGGGTTTATTAGCATC
GGGTCAAACACAGGCTTTCTGGTTCGGATCGAGCGGCTATACGCAGACTAAGTACCCGATCG
TGTTGGGCCACGGTATGCTTGGATTCGATTCGATTCTGGGGGTAGACTATTGGTACGGAATT
CCGGCCGCCTTGCGCCGTGACGGCGCTTCTGTCTATGTAACCGAGGTCTCTCAATTGGACAC
AAGCGAGGCTCGTGGCGAACAATTATTACAGCAAGTCGAAGACATTGTAGCCATCTCAGGCA
AAGGCAAAGTCAACTTAATCGGCCACAGTCACGGGGGACCAACCACTCGCTACGTGGCCGCG
GTACGCCCAGACTTGGTTGCGTCAGTAACGTCTGTTGGAGCCCCGCATAAAGGATCCGCAAC
GGCCGACTTTCTTAAAGGGATCAGTGAAGGTCCAGCTGGCCCAGTCGCTACTCCAGTATTGG
TAGGCATTGTAAATGGGCTGGGCGCACTGATTAACTTCCTTTCAGGATCGAGCTCAACGACG
CCCCAAAACGCGTTAGGATCCTTAGAATCTCTTAATTCCGAAGGTGCAGCACGCTTTAATGC
TAAGTTTCCGCAGGGCATTCCTACCTCTGCATGCGGAGAAGGCGCATATTCTGTGAACGGCG
TCCGTTATTACTCATGGTCAGGGACAAGTCCCCTGACGAACGTACTGGACCCCTCGGACTTG
TTATTAGGCGCATCGAGTTTAACATTCGGGAGCGAGGCAAACGATGGCTTAGTAGGTCGTTG
CAGCAGCCGTATGGGCCAAGTGATTCGCGACAACTACCGTATGAACCATCTTGATGAAGTGA
ACCAGACATTGGGTCTTACGAGTCTGTTCGAGACAGACCCGGTGACGGTTTACCGCCAACAT
GCAAACCGTCTTAAAAACGCTGGCCTGTAGAGATGAATACGTATACCAGGTGTCCTGTGAGC
AGCGAAAGCCTAAACGGG [SEQ ID NO: 14]
Left-OR2-OR1_sfGFP gBlock, including EcoRI, HindIII, EcoRV and NotI restriction sites,
OR2-OR1 promoter, T7g10 leader sequence, sfGFP sequence and T500 terminator sequence:
GCGCGAAGTTATCCTACGAATTCGTCAAAGCTTTAACACCGTGCGTGTTGACAATTTTACCT
CTGGCGGTGATAATGGTTGCGATATCAGCTAGCAATAATTTTGTTTAACTTTAAGAAGGAGA
TATACCATGAGCAAAGGAGAAGAACTTTTCACTGGAGTTGTCCCAATTCTTGTTGAATTAGA
TGGTGATGTTAATGGGCACAAATTTTCTGTCCGTGGAGAGGGTGAAGGTGATGCTACAAACG
GAAAACTCACCCTTAAATTTATTTGCACTACTGGAAAACTACCTGTTCCGTGGCCAACACTT
GTCACTACTCTGACCTATGGTGTTCAATGCTTTTCCCGTTATCCGGATCACATGAAACGTCA
TGACTTTTTCAAGAGTGCCATGCCTGAAGGTTATGTACAGGAACGCACTATATCTTTCAAAG
ATGACGGGACCTACAAGACGCGTGCTGAAGTCAAGTTTGAAGGTGATACCCTTGTTAATCGT
ATCGAGTTAAAGGGTATTGATTTTAAAGAAGATGGAAACATTCTTGGACACAAACTCGAGTA
CAACTTTAACTCACACAATGTATACATCACGGCAGACAAACAAAAGAATGGAATCAAAGCTA
ACTTCAAAATTCGCCACAACGTTGAAGATGGTTCCGTTCAACTAGCAGACCATTATCAACAA
AATACTCCAATTGGCGATGGCCCTGTCCTTTTACCAGACAACCATTACCTGTCGACACAATC
TGTCCTTTCGAAAGATCCTAACGAAAAGCGTGACCACATGGTCCTTCTTGAGTTTGTAACTG
CTGCTGGGATTACACATGGCATGGATGAGCTCTACAAACCGAGCTCGAGCAAAGCCCGCCGA
AAGGCGGGCTTTTCTGTGGGCATGCTGAGCGATATCAAGCTTGCGGCCGCACCCAGCAGCAG
GATGTAGTCCTGTAACCCACCA [SEQ ID NO: 15]
Left-IbpA_sfGFP gBlock, including EcoRI, HindIII, EcoRV and NotI restriction sites,
IbpA promoter, T7g10 leader sequence, sfGFP sequence and T500 terminator sequence:
GCGCGAAGTTATCCTACGAATTCGTCAAAGCTTAAAATAACATCATCATTACGTCGCACTGT
GGCGGCTATCGCACTTTAACGTTTCGTGCTGCCCCCTCAGTCTATGCAATAGACCATAAACT
GCAAAAAAAAGTCCGCTGATAAGGCTTGAAAAGTTCATTTCCAGACCCATTTTTACATCGGC
TAGCAATAATTTTGTTTAACTTTAAGAAGGAGATATACCATGAGCAAAGGAGAAGAACTTTT
CACTGGAGTTGTCCCAATTCTTGTTGAATTAGATGGTGATGTTAATGGGCACAAATTTTCTG
TCCGTGGAGAGGGTGAAGGTGATGCTACAAACGGAAAACTCACCCTTAAATTTATTTGCACT
ACTGGAAAACTACCTGTTCCGTGGCCAACACTTGTCACTACTCTGACCTATGGTGTTCAATG
CTTTTCCCGTTATCCGGATCACATGAAACGTCATGACTTTTTCAAGAGTGCCATGCCTGAAG
GTTATGTACAGGAACGCACTATATCTTTCAAAGATGACGGGACCTACAAGACGCGTGCTGAA
GTCAAGTTTGAAGGTGATACCCTTGTTAATCGTATCGAGTTAAAGGGTATTGATTTTAAAGA
AGATGGAAACATTCTTGGACACAAACTCGAGTACAACTTTAACTCACACAATGTATACATCA
CGGCAGACAAACAAAAGAATGGAATCAAAGCTAACTTCAAAATTCGCCACAACGTTGAAGAT
GGTTCCGTTCAACTAGCAGACCATTATCAACAAAATACTCCAATTGGCGATGGCCCTGTCCT
TTTACCAGACAACCATTACCTGTCGACACAATCTGTCCTTTCGAAAGATCCTAACGAAAAGC
GTGACCACATGGTCCTTCTTGAGTTTGTAACTGCTGCTGGGATTACACATGGCATGGATGAG
CTCTACAAACCGAGCTCGAGCAAAGCCCGCCGAAAGGCGGGCTTTTCTGTGGGCATGCTGAG
CGATATCAAGCTTGCGGCCGCACCCAGCAGCAGGATGTAGTCCTGTAACCCACCA [SEQ ID
NO: 16]
Left-OtsB_sfGFP gBlock, including EcoRI, HindIII and NotI restriction sites, OtsB
promoter, a 5′ untranslated region (UTR), sfGFP sequence and T500 terminator sequence.
GCGCGAAGTTATCCTACGAATTCGTCAAAGCTTTGCTTTTTTCTGCAACAATTTAGCGTTTT
TTCCCACCATAGCCAACCGCCATAACGGTTGGCTGTTCTTCGTTGCAAATGGCGACCCCCGT
CACACTGTCTATACTTACATGTCTGTAAAGCGCGTTCTGCGCAACACAATAAGAAAAGAGAA
GGAGGAGAACCGGATGAGCAAAGGAGAAGAACTTTTCACTGGAGTTGTCCCAATTCTTGTTG
AATTAGATGGTGATGTTAATGGGCACAAATTTTCTGTCCGTGGAGAGGGTGAAGGTGATGCT
ACAAACGGAAAACTCACCCTTAAATTTATTTGCACTACTGGAAAACTACCTGTTCCGTGGCC
AACACTTGTCACTACTCTGACCTATGGTGTTCAATGCTTTTCCCGTTATCCGGATCACATGA
AACGTCATGACTTTTTCAAGAGTGCCATGCCTGAAGGTTATGTACAGGAACGCACTATATCT
TTCAAAGATGACGGGACCTACAAGACGCGTGCTGAAGTCAAGTTTGAAGGTGATACCCTTGT
TAATCGTATCGAGTTAAAGGGTATTGATTTTAAAGAAGATGGAAACATTCTTGGACACAAAC
TCGAGTACAACTTTAACTCACACAATGTATACATCACGGCAGACAAACAAAAGAATGGAATC
AAAGCTAACTTCAAAATTCGCCACAACGTTGAAGATGGTTCCGTTCAACTAGCAGACCATTA
TCAACAAAATACTCCAATTGGCGATGGCCCTGTCCTTTTACCAGACAACCATTACCTGTCGA
CACAATCTGTCCTTTCGAAAGATCCTAACGAAAAGCGTGACCACATGGTCCTTCTTGAGTTT
GTAACTGCTGCTGGGATTACACATGGCATGGATGAGCTCTACAAACCGAGCTCGAGCAAAGC
CCGCCGAAAGGCGGGCTTTTCTGTGGGCATGCTGAGCAAGCTTGCGGCCGCACCCAGCAGCA
GGATGTAGTCCTGTAACCCACCA [SEQ ID NO: 17]
Right-OR2-OR1_mScarlet gBlock, including XbaI, NotI, EcoRV and EcoRI restriction sites,
OR2-OR1 promoter, T7g10 leader sequence, mScarlet sequence, and T500 terminator
sequence:
TGTAGTCCTGTAACCCACCATCTAGAGCGGCCGCTAACACCGTGCGTGTTGACAATTTTACC
TCTGGCGGTGATAATGGTTGCGATATCAGCTAGCAATAATTTTGTTTAACTTTAAGAAGGAG
ATATACCATGGTCAGCAAAGGAGAAGCGGTGATCAAAGAGTTCATGCGTTTTAAAGTGCATA
TGGAGGGAAGCATGAATGGCCATGAGTTTGAAATCGAGGGGGAAGGGGAAGGACGCCCATAT
GAAGGCACACAGACGGCAAAATTGAAGGTGACCAAAGGTGGGCCGCTGCCTTTTTCGTGGGA
TATTCTGTCTCCCCAGTTTATGTACGGTTCTCGCGCATTCATCAAGCACCCCGCTGATATTC
CAGATTACTATAAGCAATCTTTTCCAGAGGGTTTCAAATGGGAGCGTGTTATGAACTTCGAG
GATGGCGGAGCTGTTACGGTTACTCAGGATACCTCGCTGGAGGATGGTACGCTGATCTATAA
AGTAAAGTTACGCGGCACGAATTTCCCTCCAGACGGTCCTGTCATGCAGAAAAAAACGATGG
GTTGGGAAGCGTCAACGGAGCGTTTGTACCCGGAAGATGGCGTGTTGAAAGGTGATATTAAG
ATGGCTCTTCGCCTTAAGGATGGGGGGCGCTACTTAGCTGATTTCAAAACCACATACAAAGC
GAAGAAGCCAGTACAGATGCCTGGTGCGTACAATGTTGACCGCAAATTGGACATCACAAGTC
ACAATGAGGATTACACTGTGGTCGAGCAGTATGAGCGTAGTGAAGGTCGCCACTCTACGGGC
GGAATGGATGAGCTGTACAAGTAACCGAGCTCGAGCAAAGCCCGCCGAAAGGCGGGCTTTTC
TGTGGGGATATCTCTAGACAGAATTCTGCTGAGCGTACCTGT [SEQ ID NO: 18]
Final SOE-Merged-Left-OR2-OR1_sfGFP-Right-OR2-OR1_mScarlet:
GCGCGAAGTTATCCTACGAATTCGTCAAAGCTTTAACACCGTGCGTGTTGACAATTTTACCT
CTGGCGGTGATAATGGTTGCAGCTAGCAATAATTTTGTTTAACTTTAAGAAGGAGATATACC
ATGAGCAAAGGAGAAGAACTTTTCACTGGAGTTGTCCCAATTCTTGTTGAATTAGATGGTGA
TGTTAATGGGCACAAATTTTCTGTCCGTGGAGAGGGTGAAGGTGATGCTACAAACGGAAAAC
TCACCCTTAAATTTATTTGCACTACTGGAAAACTACCTGTTCCGTGGCCAACACTTGTCACT
ACTCTGACCTATGGTGTTCAATGCTTTTCCCGTTATCCGGATCACATGAAACGTCATGACTT
TTTCAAGAGTGCCATGCCTGAAGGTTATGTACAGGAACGCACTATATCTTTCAAAGATGACG
GGACCTACAAGACGCGTGCTGAAGTCAAGTTTGAAGGTGATACCCTTGTTAATCGTATCGAG
TTAAAGGGTATTGATTTTAAAGAAGATGGAAACATTCTTGGACACAAACTCGAGTACAACTT
TAACTCACACAATGTATACATCACGGCAGACAAACAAAAGAATGGAATCAAAGCTAACTTCA
AAATTCGCCACAACGTTGAAGATGGTTCCGTTCAACTAGCAGACCATTATCAACAAAATACT
CCAATTGGCGATGGCCCTGTCCTTTTACCAGACAACCATTACCTGTCGACACAATCTGTCCT
TTCGAAAGATCCTAACGAAAAGCGTGACCACATGGTCCTTCTTGAGTTTGTAACTGCTGCTG
GGATTACACATGGCATGGATGAGCTCTACAAACCGAGCTCGAGCAAAGCCCGCCGAAAGGCG
GGCTTTTCTGTGGGCATGCTGAGCAAGCTTGCGGCCGCACCCAGCAGCAGGATGTAGTCCTG
TAACCCACCATCTAGAGCGGCCGCTAACACCGTGCGTGTTGACAATTTTACCTCTGGCGGTG
ATAATGGTTGCAGCTAGCAATAATTTTGTTTAACTTTAAGAAGGAGATATACCATGGTCAGC
AAAGGAGAAGCGGTGATCAAAGAGTTCATGCGTTTTAAAGTGCATATGGAGGGAAGCATGAA
TGGCCATGAGTTTGAAATCGAGGGGGAAGGGGAAGGACGCCCATATGAAGGCACACAGACGG
CAAAATTGAAGGTGACCAAAGGTGGGCCGCTGCCTTTTTCGTGGGATATTCTGTCTCCCCAG
TTTATGTACGGTTCTCGCGCATTCATCAAGCACCCCGCTGATATTCCAGATTACTATAAGCA
ATCTTTTCCAGAGGGTTTCAAATGGGAGCGTGTTATGAACTTCGAGGATGGCGGAGCTGTTA
CGGTTACTCAGGATACCTCGCTGGAGGATGGTACGCTGATCTATAAAGTAAAGTTACGCGGC
ACGAATTTCCCTCCAGACGGTCCTGTCATGCAGAAAAAAACGATGGGTTGGGAAGCGTCAAC
GGAGCGTTTGTACCCGGAAGATGGCGTGTTGAAAGGTGATATTAAGATGGCTCTTCGCCTTA
AGGATGGGGGGCGCTACTTAGCTGATTTCAAAACCACATACAAAGCGAAGAAGCCAGTACAG
ATGCCTGGTGCGTACAATGTTGACCGCAAATTGGACATCACAAGTCACAATGAGGATTACAC
TGTGGTCGAGCAGTATGAGCGTAGTGAAGGTCGCCACTCTACGGGCGGAATGGATGAGCTGT
ACAAGTAACCGAGCTCGAGCAAAGCCCGCCGAAAGGCGGGCTTTTCTGTGGGTCTAGACAGA
ATTCTGCTGAGCGTACCTGT [SEQ ID NO: 19]
SOE/NEBuilder result-PCLase-1:
GCGCGAAGTTATCCTACGAATTCGTCAAAGCTTTAACACCGTGCGTGTTGACAATTTTACCT
CTGGCGGTGATAATGGTTGCGATATCAGCTAGCAATAATTTTGTTTAACTTTAAGAAGGAGA
TATACCATGCTTGATCTGAAATTGGGAGGCTTGGCTGCTGCTTGTTTGCTGGTCTGTTCTAC
GGCACTTGCCGCACCTTTACCAGACACCCCTGGGGCGCCTTTTCCAGCAGTTGCTAACTTCG
ATCGTTCTGGACCTTACGCTACTAGCAATCAATCCGAAGGTCCCTCATGTCGCATCTACCGT
CCCTCTAATTTGGGACAGGGGGGTGTGCGTCACCCAGTAATTTTGTGGGGAAACGGTACGGG
CACAGGTCCCTCCACCTATGCGGGCCTTTTATCCCATTGGGCCTCGCATGGCTTCGTCGTAG
CGGCGGCTGAGACGTCGAATGCCGGTACAGGGCGCGAAATGCTGGCCTGTTTGGACTATCTT
GTGCGCGAAAATGATAATCCCTATGGGACTTATGCTGGTAAGTTGAACACAGGACGTGTGGG
GACATCCGGTCACTCCCAAGGCGGCGGCGGTTCAATTATGGCGGGGCAGGATACCCGTGTGC
GTACGACGGCACCAATTCAACCTTACACCATTGGACTGGGCCATGATAGCGCCTCACAACGT
CGCCAACAGGGGCCAATGTTCCTTATGTCCGGTGGCGGCGACACTATCGCTATCCCCTATCT
TAACGCGCAGCCAGTGTATCTTCGCGCCAATGTACCCGTTTTCTGGGGCGAGCGCCGTTACG
TTTCTCATTTCGAGCCCGTGGGAGACGGTGGTGCATATCGTGGCCCCAGCACGGCCTGGTTT
CGTTTCCAGTTGATGGACGATCAGTCAGCGCGTGGTACTTTTTATGGGACTCTGTGCTCCCT
GTGTTCCTCATTATTGTGGAGTGTCGAGCGCCGTGGCTTCTAGAGATGAATACGTATACCAG
GTGTCCTGTGAGCAGCGAAAGCCTAAACGGGTCTAGAGATTAAAGAGGAGAAATACTAGATG
AGCAAAGGAGAAGAACTTTTCACTGGAGTTGTCCCAATTCTTGTTGAATTAGATGGTGATGT
TAATGGGCACAAATTTTCTGTCCGTGGAGAGGGTGAAGGTGATGCTACAAACGGAAAACTCA
CCCTTAAATTTATTTGCACTACTGGAAAACTACCTGTTCCGTGGCCAACACTTGTCACTACT
CTGACCTATGGTGTTCAATGCTTTTCCCGTTATCCGGATCACATGAAACGTCATGACTTTTT
CAAGAGTGCCATGCCTGAAGGTTATGTACAGGAACGCACTATATCTTTCAAAGATGACGGGA
CCTACAAGACGCGTGCTGAAGTCAAGTTTGAAGGTGATACCCTTGTTAATCGTATCGAGTTA
AAGGGTATTGATTTTAAAGAAGATGGAAACATTCTTGGACACAAACTCGAGTACAACTTTAA
CTCACACAATGTATACATCACGGCAGACAAACAAAAGAATGGAATCAAAGCTAACTTCAAAA
TTCGCCACAACGTTGAAGATGGTTCCGTTCAACTAGCAGACCATTATCAACAAAATACTCCA
ATTGGCGATGGCCCTGTCCTTTTACCAGACAACCATTACCTGTCGACACAATCTGTCCTTTC
GAAAGATCCTAACGAAAAGCGTGACCACATGGTCCTTCTTGAGTTTGTAACTGCTGCTGGGA
TTACACATGGCATGGATGAGCTCTACAAACCGAGCTCGAGCAAAGCCCGCCGAAAGGCGGGC
TTTTCTGTGGGCATGCTGAGCGATATCAAGCTTGCGGCCGCACCCAGCAGCAGGATGTAGTC
CTGTAACCCACCA [SEQ ID NO: 20]
SOE/NEBuilder result-PCLase-2:
GCGCGAAGTTATCCTACGAATTCGTCAAAGCTTTAACACCGTGCGTGTTGACAATTTTACCT
CTGGCGGTGATAATGGTTGCGATATCAGCTAGCAATAATTTTGTTTAACTTTAAGAAGGAGA
TATACCATGAAGAAGAACAAAACCCTTTTGGCATTGTGCCTGGGCACGGGTTTATTAGCATC
GGGTCAAACACAGGCTTTCTGGTTCGGATCGAGCGGCTATACGCAGACTAAGTACCCGATCG
TGTTGGGCCACGGTATGCTTGGATTCGATTCGATTCTGGGGGTAGACTATTGGTACGGAATT
CCGGCCGCCTTGCGCCGTGACGGCGCTTCTGTCTATGTAACCGAGGTCTCTCAATTGGACAC
AAGCGAGGCTCGTGGCGAACAATTATTACAGCAAGTCGAAGACATTGTAGCCATCTCAGGCA
AAGGCAAAGTCAACTTAATCGGCCACAGTCACGGGGGACCAACCACTCGCTACGTGGCCGCG
GTACGCCCAGACTTGGTTGCGTCAGTAACGTCTGTTGGAGCCCCGCATAAAGGATCCGCAAC
GGCCGACTTTCTTAAAGGGATCAGTGAAGGTCCAGCTGGCCCAGTCGCTACTCCAGTATTGG
TAGGCATTGTAAATGGGCTGGGCGCACTGATTAACTTCCTTTCAGGATCGAGCTCAACGACG
CCCCAAAACGCGTTAGGATCCTTAGAATCTCTTAATTCCGAAGGTGCAGCACGCTTTAATGC
TAAGTTTCCGCAGGGCATTCCTACCTCTGCATGCGGAGAAGGCGCATATTCTGTGAACGGCG
TCCGTTATTACTCATGGTCAGGGACAAGTCCCCTGACGAACGTACTGGACCCCTCGGACTTG
TTATTAGGCGCATCGAGTTTAACATTCGGGAGCGAGGCAAACGATGGCTTAGTAGGTCGTTG
CAGCAGCCGTATGGGCCAAGTGATTCGCGACAACTACCGTATGAACCATCTTGATGAAGTGA
ACCAGACATTGGGTCTTACGAGTCTGTTCGAGACAGACCCGGTGACGGTTTACCGCCAACAT
GCAAACCGTCTTAAAAACGCTGGCCTGTAGAGATGAATACGTATACCAGGTGTCCTGTGAGC
AGCGAAAGCTCTAGAGATTAAAGAGGAGAAATACTAGATGAGCAAAGGAGAAGAACTTTTCA
CTGGAGTTGTCCCAATTCTTGTTGAATTAGATGGTGATGTTAATGGGCACAAATTTTCTGTC
CGTGGAGAGGGTGAAGGTGATGCTACAAACGGAAAACTCACCCTTAAATTTATTTGCACTAC
TGGAAAACTACCTGTTCCGTGGCCAACACTTGTCACTACTCTGACCTATGGTGTTCAATGCT
TTTCCCGTTATCCGGATCACATGAAACGTCATGACTTTTTCAAGAGTGCCATGCCTGAAGGT
TATGTACAGGAACGCACTATATCTTTCAAAGATGACGGGACCTACAAGACGCGTGCTGAAGT
CAAGTTTGAAGGTGATACCCTTGTTAATCGTATCGAGTTAAAGGGTATTGATTTTAAAGAAG
ATGGAAACATTCTTGGACACAAACTCGAGTACAACTTTAACTCACACAATGTATACATCACG
GCAGACAAACAAAAGAATGGAATCAAAGCTAACTTCAAAATTCGCCACAACGTTGAAGATGG
TTCCGTTCAACTAGCAGACCATTATCAACAAAATACTCCAATTGGCGATGGCCCTGTCCTTT
TACCAGACAACCATTACCTGTCGACACAATCTGTCCTTTCGAAAGATCCTAACGAAAAGCGT
GACCACATGGTCCTTCTTGAGTTTGTAACTGCTGCTGGGATTACACATGGCATGGATGAGCT
CTACAAACCGAGCTCGAGCAAAGCCCGCCGAAAGGCGGGCTTTTCTGTGGGCATGCTGAGCG
ATATCAAGCTTGCGGCCGCACCCAGCAGCAGGATGTAGTCCTGTAACCCACCA [SEQ ID
NO: 21]

### DUF1646_sfGFP:

The sequence is the same as the OR1/2 sfGFP gblock but with a DUF1646 sequence in between the promoter and start codon (underlined). The DUF riboswitch sequence is from (White N. et al supra) and confirmed using (Clostridium acetobutylicum strain LJ4 chromosome-GenBank: CP030018.1). The sequence also uses same primers as original OR1/2 sfGFP gblock and joins onto Right_mScarlet in the same manner.

[SEQ ID NO: 22]
GCGCGAAGTTATCCTACGAATTCGTCAAAGCTTTAACACCGTGCGTGTTGACAATTTTACCT
CTGGCGGTGATAATGGTTGCGATATCATTATATTAAATAAAATATTTTAACATTTCGGTTGA
GCAAATTTATATTTGTTTTCAAGAGCTTTGAATTTCTGGGGTGGGAAATTCCTTAAAAGAGG
AATATCCTACCTTTTTTGCGTACAAAATATTATTTGGAGGTGTGATTTATGAGCAAAGGAGA
AGAACTTTTCACTGGAGTTGTCCCAATTCTTGTTGAATTAGATGGTGATGTTAATGGGCACA
AATTTTCTGTCCGTGGAGAGGGTGAAGGTGATGCTACAAACGGAAAACTCACCCTTAAATTT
ATTTGCACTACTGGAAAACTACCTGTTCCGTGGCCAACACTTGTCACTACTCTGACCTATGG
TGTTCAATGCTTTTCCCGTTATCCGGATCACATGAAACGTCATGACTTTTTCAAGAGTGCCA
TGCCTGAAGGTTATGTACAGGAACGCACTATATCTTTCAAAGATGACGGGACCTACAAGACG
CGTGCTGAAGTCAAGTTTGAAGGTGATACCCTTGTTAATCGTATCGAGTTAAAGGGTATTGA
TTTTAAAGAAGATGGAAACATTCTTGGACACAAACTCGAGTACAACTTTAACTCACACAATG
TATACATCACGGCAGACAAACAAAAGAATGGAATCAAAGCTAACTTCAAAATTCGCCACAAC
GTTGAAGATGGTTCCGTTCAACTAGCAGACCATTATCAACAAAATACTCCAATTGGCGATGG
CCCTGTCCTTTTACCAGACAACCATTACCTGTCGACACAATCTGTCCTTTCGAAAGATCCTA
ACGAAAAGCGTGACCACATGGTCCTTCTTGAGTTTGTAACTGCTGCTGGGATTACACATGGC
ATGGATGAGCTCTACAAACCGAGCTCGAGCAAAGCCCGCCGAAAGGCGGGCTTTTCTGTGGG
CATGCTGAGCGATATCAAGCTTGCGGCCGCACCCAGCAGCAGGATGTAGTCCTGTAACCCAC
CA

### PCR Protocol

Each 1000 ng gBlock was suspended in 10 μL ultrapure water (UPW) and the following reaction mixture was prepared:

| | |
|---|---|
| Nuclease Free Water/UPW | 63 μL |
| 5X Q5 Buffer (NEB - #B9027S) | 20 μL |
| 10 mM dNTPs (NEB- #N0446S) | 4 μL |
| 10 μM Forward Primer | 5 μL |
| 10 μM Reverse Primer | 5 μL |
| DNA Template (100 ng/μL) | 2 μL |
| Q5 Taq Polymerase (NEB - #M0491S) | 1 μL |

The following thermocycler program (Applied Biosystems ProFlex PCR system) was prepared and run:

| Temperature | Time | Cycles |
|---|---|---|
| 98° C. | 1 Minute | 1x |
| 98° C. | 10 Seconds | 12x |
| 62° C. | 30 Seconds | |
| 72° C. | 4 Minutes | |
| 72° C. | 10 Minutes | 1x |

Products were then purified using Qiagen QIAquick PCR purification columns hand (Qiagen-#28106) using a centrifuge (Hitachi-#CT15RE). 500 μL PB buffer was added to 100 μL PCR mixture and mixed by pipetting. The 600 μL mixture was loaded into a spin column and centrifuged at 17,000 g for 1 minute, the flow-through discarded. The spin column was loaded with 600 μL PE wash buffer with ethanol, centrifuged at 17,000 g for 1 minute, and the flow-through discarded. The empty column was centrifuged at 17,000 g for 1 minute to remove residual wash buffer. The PCR purification column was transferred into a fresh microcentrifuge tube, 25 μL of ultrapure water pipetted into the centre of the column membrane and left for 5 minutes. The empty column was centrifuged at 17,000 g for 1 minute to extract DNA before quantifying via Nanodrop UV-vis.

Sequences were joined together via Splicing by Overlap Extension (SOEing; Horton R. M. et al (2013) BioTechniques, 54 (3), 129-133). Each gBlock was designed with overlapping, complementary sequences in the primer region so that they could be joined by performing PCR for several cycles without primers. sfGFP was always upstream (left) of the mScarlet part (right). As mScarlet was always constitutively active, having OR1/2_mScarlet downstream meant that any sfGFP expression was only due to OtsB expression when testing salt responsive strains. The PCR process was repeated with the following changes:

| | |
|---|---|
| Nuclease Free Water/UPW | 62 μL |
| 5X Q5 Buffer (NEB - #B9027S) | 20 μL |
| 10 mM dNTPs (NEB- #N0446S) | 4 μL |
| “Left” DNA Template | 1 μL |
| “Right” DNA Template | 1 μL |
| Q5 Taq Polymerase (NEB - #M0491S) | 1 μL |

This mixture was run for 4 cycles before adding 5 μL of each primer to amplify the spliced products by completing the remaining cycles of the PCR program.

For the PCLase and DUF1646 assemblies, gBlock sequences were spliced together using NEBuilder HiFi DNA Assembly Cloning kit (NEB-#E5520S) using the same complementary end regions required for SOEing. The reaction mixture was set up as follows:

| | |
|---|---|
| Nuclease Free Water/UPW | 4 μL |
| “Left” DNA template (100 ng/μL) | 0.5 μL |
| “Right” DNA template (100 ng/μL) | 0.5 μL |
| NEBuilder Mastermix (NEB - #M5520A) | 5 μL |

The mixture was incubated at 50° C. for 15 minutes before directly amplifying via PCR, preparing the following reaction mixture:

| | | |
|---|---|---|
| Nuclease Free Water/UPW | 27 | μL |
| 5X Q5 Buffer (NEB - #B9027S) | 10 | μL |
| GC Enhancer (NEB - #B9028A) | 5 | μL |
| 10 mM dNTPs (NEB- #N0446S) | 2 | μL |
| 10 μM Forward Primer | 2.5 | μL |
| 10 μM Reverse Primer | 2.5 | μL |
| Completed NEBuilder reaction mixture | 0.5 | μL |
| Q5 Taq Polymerase (NEB - #M0491S) | 0.5 | μL |

The thermocycler program and purification steps were the same as previously described.

Spliced products were then digested with restriction enzymes (specifically either EcoRI-HF (NEB-#R3101S) or HindIII (NEB-#R0104S) depending on the insert) to produce sticky ends to either end of the SOEing products. pUC19 plasmid vector (NEB-#N3041S) was also digested using EcoRI-HF or HindIII. Digested products and plasmid were then ligated together for transformation into E. coli.

### Restriction Digest Protocol:

The following solution mixture was prepared:

| | | |
|---|---|---|
| Purified DNA | 2000 | ng |
| 10X CutSmart buffer (NEB- #B6004S) | 10 | μL |
| EcoRI-HF(NEB - #R3101S), | 2 | μL |
| HindIII (NEB - #R0104S) | | |

| | |
|---|---|
| UPW | Up to 100 μL |

The mixture was placed in a thermocycler to incubate for 1 hour at 37° C., then heat inactivated at 65° C. for 20 minutes. The mixture was purified using Qiagen QIAquick PCR purification columns and eluted in 30 μL ultrapure water.

### Ligation Protocol:

The following solution mixture was prepared:

| | |
|---|---|
| Digested pUC19 | 5 μL |
| Digested insert | 10 μL |
| T4 Buffer (NEB - #B0202S) | 10 μL |
| T4 Ligase (NEB - #M0202S) | 4 μL |
| UPW | 71 μL |

The mixture was placed in a thermocycler to incubate for 2 hours at 24° C., then heat inactivated at 65° C. for 20 minutes. The mixture was purified using Qiagen QIAquick PCR purification columns and eluted in 30 μL ultrapure water.

Ligated products were then analysed via gel electrophoresis to compare and confirm product sizes (see below and FIG. 1). The final constructs (ligated pUC19+insert) bands were then excised and purified via gel extraction (Monarch DNA Gel Extraction Kit (NEB-#T1020S)) to provide pure plasmid with the correct insert.

The band from the gel was excised using a scalpel and a blue-light Safe Imager (Invitrogen-#G6600UK) to visualise the bands. The band was removed as a small rectangular block of agarose, taking care to avoid including unwanted bands. The extracted agarose block was placed in a 1.5 mL microcentrifuge tube and weighed before 4 volumes of Monarch Gel Dissolving Buffer were added to the tube with the gel slice (e.g., 400 μl buffer per 100 mg agarose). The sample was incubated between at 50° C. for 10 minutes until completely melted and dissolved and then loaded into a gel extraction spin column inserted into a collection tube and centrifuged for 1 minute at 16,000 g. The flow through was discarded. 200 μL DNA wash buffer was added to the spin column and centrifuged for 1 minute at 16,000 g. The flow through was discarded. A second wash step was carried out by adding 200 μL DNA wash buffer to the spin column, centrifuging for 1 minute at 16,000 g and the flow through discarded. The spin column was transferred to a fresh 1.5 mL microcentrifuge tube. 10 μL UPW was pipetted directly into the spin column membrane and left for 5 minutes before centrifugation for 1 minute at 16,000 g to extract purified plasmid DNA for transformation.

The prepared plasmid was then transformed into DH5a E. coli (NEB-#C2987H) using a heat shock protocol. Heat shock protocol:

A tube of NEB 5-alpha Competent E. coli cells was thawed on ice for 10 minutes. 1-5 μl containing 1 μg-100 ng of plasmid DNA was added to the cell mixture, the tube carefully flicked 4-5 times to mix cells and DNA and placed on ice for 30 minutes.

Heat shock was carried out at exactly 42° C. for exactly 30 seconds, the mixture then placed on ice for 5 minutes before 950 μl of room temperature Super Optimal broth with Catabolite repression medium (SOC medium) was pipetted into the mixture. The resulting mixture was placed on ice for 5 minutes, warmed at 37° C. for 60 minutes before being shaken vigorously at 250 rpm.

Selection plates were warmed to 37° C. The cellular mixture was mixed thoroughly by flicking the tube and inverting, before several 10-fold serial dilutions in SOC medium were performed. 50 μl of each dilution was spread onto a selection plate and incubated overnight at 37° C. Alternatively, the cells were incubated at 30° C. for 24-36 hours or 25° C. for 48 hours.

pUC19 also contains an ampicillin resistance gene. LB agar plates were prepared with 10 g LB powder (VWR-#84649.05) and 7.5 g agar (VWR-#20767.23) in 500 mL UPW. This mixture was then autoclaved, cooled (but hot enough to still be liquid and poured into plates), before adding 500 μL 100 mg/mL ampicillin (Fisher Scientific—#10419313). Liquid LB media+ampicillin for culturing was prepared in the same way but without agar.

Colonies were picked by using the blue-light safe imager to visualise fluorescent colonies on the agar plates. E. coli were then grown from individual colonies in 96 well plates (48 strains per insert variant) and cultured in LB media+ampicillin overnight at 37° C. (shaking incubator, 300 rpm) before being scanned for successful expression of both sfGFP and mScarlet using a BMG Clariostar plate reader, with spectral scans set for each protein. (sfGFP: Ex=470 nm, Em=490 nm-560 nm mScarlet: Ex=555 nm, Em=590 nm−670 nm). A strain from each insert that demonstrated both proteins being expressed was then cultured in bulk in 4×100 mL Erlenmeyer flasks of LB+ampicillin. These flasks were incubated at 37° C. (shaking incubator, 300 rpm) for 2 days.

### Blue/White Screening and Colony PCR

For non-fluorescent inserts (such as PCLases), blue/white colony screening was used to determine successful transformants. When preparing LB/amp/agar plates, 5 mL of 100 mM Isopropyl β-D-1-thiogalactopyranoside (IPTG) (Sigma-#16758-1G) and 5 mL of 20 mg/mL X-gal (VWR-#437132J) in DMSO (Fisher Scientific-#D/4121/PB08) was added to 500 mL liquid LB Agar after autoclaving before pouring. Colonies which have successful been heat-shocked with pUC19 will contain β-galactosidase, expression of which is induced by IPTG and can then catalyse the breakdown of X-gal to produce a blue colour. Insertion of desired genes via restriction digest and ligation was targeted within the β-galactosidase gene, interrupting the coding sequence and preventing it from being expressed and breaking down x-gal, making successful transformants appear white instead of blue. White colonies were picked, cultured in LB media and colony PCR used to confirm the presence of inserts using the following reaction mixture:

| | | |
|---|---|---|
| Nuclease Free Water/UPW | 4.4 | μL |
| 5X Q5 Buffer (NEB - #B9027S) | 2 | μL |
| GC Enhancer (NEB - #B9028A) | 1 | μL |
| 10 mM dNTPs (NEB- #N0446S) | 0.5 | μL |
| 10 μM Forward Primer | 0.5 | μL |
| 10 μM Reverse Primer | 0.5 | μL |
| Q5 Taq Polymerase (NEB - #M0491S) | 0.1 | μL |
| Bacterial sample in ultrapure water | 1 | μL |

Primers used were the “Reverse” of the “Left” gBlock and the “Forward” of the “Right” gBlock, meaning that the polymerase moved outwardly from the insert, amplifying the entire ~1.8 kb insert and ~2.6 kb plasmid as a ~4.4 kb linear amplicon. This larger amplicon also led to a slight increase in dNTP concentration being used compared to other PCR reactions. The following thermocycler program was used:

| Temperature | Time | Cycles |
|---|---|---|
| 98° C. | 15 Minutes | 1x |
| 98° C. | 10 Seconds | 30x |
| 62° C. | 30 seconds | |
| 72° C. | 5 Minutes | |
| 72° C. | 10 Minutes | 1x |

Each 10 μL of colony PCR product had 6 μL of Purple Loading Dye (NEB-#B7024S) added and mixed, and was analysed by gel electrophoresis, using 1% agarose (Invitrogen-#S33102), SYBRsafe DNA stain (Fisher-#S33102) and 1X TAE buffer (Fisher-#B49). A band at approximately 4.4 kb indicated that the transformation was successful.

### PCLase Degradation

PCL granules (Easycomposites-#MP-PCL-4) were placed on a sheet of aluminium foil on top of a heat block set at 80° C. and melted. Once molten, the granules were pressed flat with a spatula, then removed from the heat and peeled off the foil once cooled. This was done to increase the amount of surface area available during the PCL degradation reaction. Flakes were then weighed using an analytical balance (Radwag-#AS 220.R2) in sets of five flakes with the weights recorded. Flakes were then placed in 2 mL microcentrifuge tubes with 400 μL 100% ethanol (Sigma-#1.08543.0250) added to sterilise the flakes before addition to bacterial culture media. The flakes were then poured into wells in a 24-well culture plate (five flakes per well with masses known) within a biohood, with the excess ethanol pipetted off and left to air dry.

A customised LB media was prepared (“PCLase media”), designed to enhance both PCLase-1 activity and protein secretion of DH5-a E. coli. PCLase-1 activity was optimised by including 5 mM Mg²⁺ (Fisher-#12288.36) 5 mM Ca²⁺ (Fisher-#L13191.30), 1 mM Fe (III) (Sigma-#157740-100G) and adjusted to pH 8.5 using Trizma base (Sigma-#T1503-500G) (Li L. et al supra). Protein secretion was optimised by including 1% glycine (GE Healthcare-#17-1323-01), 5% sucrose (Fisher-#BP220-1), and 0.5% Tween-80

(Sigma-#P4780-100 ml) (Bao R-M. et al (2016) Protein Expression and Purification, 126, 9-15). PCLase media also included ampicillin to the same concentration as previous LB media used to protect against contamination.

Two strains of E. coli were prepared, one control and one containing PCLase-1. Two conditions were prepared, a high density of bacteria to begin the degradation as quickly as possible, and a low-density culture, intended to grow in the presence of the PCL flakes. The high-density culture was prepared using a large 50 mL stock was cultured in standard LB/amp media in an Erlenmeyer flask for 2 days at 37° C., shaking at 200 rpm, and pelleted down via centrifugation at 5000 rpm for 20 minutes. This pellet was then resuspended in 5 mL optimised PCLase media. The low-density culture was a 1:5 diluted LB media solution taken directly from the microplate liquid cultures used at the colony PCR screening stage.

Each well containing five PCL flakes had 1 mL of PCLase media added. Each condition was also accompanied by blank wells with flakes and media but no bacteria. The high-density cultures had 1 mL of pelleted and resuspended bacteria/PCLase media added instead of blank media. The low density cultured had 1 mL of PCLase media with 50 μL dilute bacteria solution added. These cultures were then allowed to grow in a static 37° C. incubator for 24 hours, with a static incubator used to allow the bacteria and flakes to settle and remain in contact with each other to promote degradation.

After 24 hours, the media was pipetted off the flakes, with samples taken to analyse via Nanodrop UV/vis spectroscopy (Thermo Scientific-#ND2000USCAN) to determine the final OD600 density values of the bacterial cultures. With the media and bacteria removed, the flakes were rinsed within their wells with 500 μL 70% ethanol, swirled to remove any bacterial mass, and the liquid pipetted off. The flakes were then rinsed again with 1 mL ultrapure water, swirled and the liquid pipetted off. A final wash step of 500 μL 100% ethanol was added, swirled, the liquid pipetted off, and left to air dry for 1 hour. Each set of flakes was then weighed again, and the masses compared to those before the degradation experiment. Each condition was tested in triplicate.

### Preparation of Bacterial/PCL Filament

Bulk volumes of bacterial culture were loaded into 50 mL falcon tubes and centrifuged at 4000 rpm for 15 minutes to pellet the bacteria. The supernatant was discarded. A mixture of 60% Polyvinyl alcohol (PVA) glue/LB media was made, with the LB media used being 2.5× concentration to maintain 1×after combination with PVA. To each pellet, 3 mL of PVA was added and mixed. 30 g of polycaprolactone pellets (Polyshape-#4336898380) were added to each falcon tube, stirred, then vortexed to coat the pellets evenly in bacterial PVA solution. The beads were then poured out and spread across plastic petri dishes, loaded into a dehydrator (HOMCOM-#800-029) and dried at 40° C. for 1 hour.

A Filastruder with a 1.75 mm nozzle was used to form the bacteria-coated PCL beads into a filament. The Filastruder was set to 65° C. with beads slowly added to avoid jamming the auger. Filament was either fed directly into an additional Filawinder device that spools formed filament immediately using a laser sensor to detect slack in the produced filament, or the Filawinder was positioned directly over an ice bath, where the rapid cooling immediately cooled and set the extruded filament at the diameter of the nozzle.

FIG. 2 shows a filament being produced using a Filastruder machine using PCL beads coated in OR2—OR1_sfGFP E. coli (no mScarlet; FIG. 2a). In FIG. 2b, the Extruder is extruding filament directly into an ice bath, where the drop in temperature solidifies the molten plastic and the hydrophobic nature of PCL allows it to slide along, keeping extrusion smooth.

### Printing of Bacterial/PCL Filament

Printing of bacterial filament was carried out using an EasyThreeD K7 direct drive 3D printer. Extrusion temperature was set to 85° C. Additional USB powered cooling fans (ELUTENG-#ELT-CDUFAN-80-EU) were positioned to cool the print and the printer stepper motor to avoid “heat creep”, where the gear of the extruder motor heats up and begins to slip through the low temperature filament. Double-sided sticky tape was applied to the print bed to help with print adhesion, being removed after the print.

In the salt response tests, filament was then printed into discs 8 mm in diameter, 1 layer (0.2 mm) thick. Printed discs were pushed to the bottoms of wells in a black 96 well fluorescence plate (Corning-#CLS3603-48EA), with half of the wells being filled with 100 μL M9 media and the other half being filled with 0.6M NaCl M9 media (Sigma-#73176-1 KG). Fluorescence was scanned using a BMG Fluorostar, with fluorescence scanned over time (sfGFP: ex=485 nm, em=520 nm, mScarlet: ex=584 nm, em=620 nm. mScarlet has limited utility plastic experiments because extruded plastics often have autofluorescent properties. In this instance PCL has an autofluorescence in the same region as mScarlet and so masks this measurement. As a result, relative sfGFP fluorescence was used in the printed salt tests.

FIG. 3 is a demonstration of a printed test cube using a E. coli/PCL filament made as described above. The block containing E. coli is dramatically more fluorescent than the control. The control PCL filament was made as described above but without the bacteria.

### Example 1: Fluorescence Microscopy of Printed Parts.

This experiment was carried out to confirm that the fluorescence in the filament is due to the biological elements introduced into the plastic, rather than any autofluorescence from the plastic.

As can be seen in FIG. 4, bacteria appeared to be mostly intact based on the shape and size of the fluorescence. For example, in FIG. 4a, streaks of green within the disc show areas with higher densities of sfGFP E. coli. In FIG. 4b, a layer-by-layer crosshatched structure produced by filament 3D printing can be seen. FIG. 4c shows individual bacteria embedded in the plastic, while FIG. 4d confirms that the structures in the plastic are sfGFP expressing bacteria.

Example 2: Filaments with Different Proteins of Interest

Polycaprolactone (PCL) beads were treated with different engineered E. coli, one producing a green fluorescent protein (sfGFP; FIG. 5 left), and the other producing a red fluorescent protein (mScarlet; FIG. 5 right). Both used the same promoter for expression, only the protein was changed. This experiment demonstrated that different proteins of interest can be selected, successfully expressed in E. coli and remain viable once added to a plastic and extruded as a filament.

Example 3: Demonstration of Salt Responsive Bacteria within Plastic.

This experiment tested a simple bacterial system at a low temperature and without any additional heat resistance steps.

E. coli were engineered with OtsB_sfGFP and OR-2—OR1 mScarlet, incorporated into plastic filament and printed into thin discs for scanning using a fluorescence plate reader, as described above. A relative increase in sfGFP fluorescence over time was used as a measure of bacterial response to environmental salinity. All discs contained bacteria. Half of the wells had additional salt (at seawater concentrations (0.6M NaCl) while the other half had standard M9 media.

As shown in FIG. 6, the addition of salt induced increased expression of sfGFP, demonstrating both that bacteria can be included within 3D printable filament and parts and that, after printing, those bacteria can perform their designed functions such as responding to environmental salinity. Errors bars in FIG. 6b are large due to the patchy nature of bacteria spread throughout the plastic and the averaging method used by the plate reader. However, this is balanced out by the large number of replicates for each set. The increase in fluorescence is far greater for those exposed to high salinity.

Example 4: The above experiment was repeated using LB media instead of M9 media to grow the bacteria before being centrifuged into pellets and resuspended in PBS for scanning. The aim of this experiment was to show that the fluorescence was not an artefact of the cell media. As shown in FIG. 7, the bacteria remain viable in a sodium chloride solution of 0.3M, and the experiment confirms that fluorescence is a result of sfGFP and mScarlet owing to the similarities of their emission spectra to other published research (https://www.fpbase.org/protein/superfolder-gfp/, https://www.fpbase.org/protein/mscarlet/)
Example 5: the experiment of Example 4 was repeated but comparing use of a riboswitch instead of a promoter, different salt concentrations, and without the step of pelleting and resuspending in PBS. Bacteria were cultured for 24 hours at 37° C., shaken at 200 rpm, and were then scanned for fluorescence using a BMG Clariostar in 1X Lennox LB media at different salt concentrations. E. coli were engineered to produce mScarlet with a constitutively active promoter as a marker of cell density, and sfGFP also using the same constitutively active promoter but including a DUF1646 Na⁺ sensitive riboswitch over the ribosome binding site. When Na⁺ ions are not present, the riboswitch structure occludes the ribosome binding site and prevents translation. When Na⁺ ions present in a sufficient concentration, the riboswitch structure is destabilised, allowing the protein to be translated.

The data, shown in FIG. 8, demonstrates that DUF1646 can be used to control sfGFP expression in response to different salt concentrations. All emission values were referenced against the peak value (595 nm) of mScarlet for each condition to show the relative changes of gene expression within the bacteria.

### Example 6: Measure of Response to Salt in OtsB_sfGFP+OR-2OR1_mScarlet.

Bacteria in M9 media were scanned for both sfGFP and mScarlet over time. Because mScarlet is constitutively active, it was used as a measure of cell density and to account for any stunting of E. coli growth caused by high salt solutions. As a result, the relative ratio of sfGFP to mScarlet within bacteria was being compared (i.e, sfGFP intensity/mScarlet intensity).

As seen in FIG. 9, the bacteria were capable of responding specifically to salt, with the concentration of salt increasing the level of sfGFP expression. FIG. 9a plots kinetics over time and shows a gradual production of sfGFP once exposed to salt, again with higher NaCl concentrations inducing sfGFP expression (BMG Fluorostar). These measurements used filters that are close but not exact for optimal sfGFP mScarlet spectra and are each normalised to “1” at the starting time point. FIG. 9b shows final endpoints of sfGFP/mScar normalised to the value of “OM” NaCl to show how the level of sfGFP expression changed with concentration. Results from a spectral scan on a BMG Clariostar using optimal excitation/emission wavelengths were used for the calculation (see graphs for exact wavelengths).

Example 7:8-Hour Kinetics of DUF 1646-sfGFP Expression

Using the same strain as in Example 5, DUF1646 Na⁺ sensitive riboswitch controlled sfGFP expression was tested in different NaCl concentrations over time. E. coli were cultured in 1.25X Lennox LB and scanned for fluorescence using a BMG Fluostar set at 37° C., shaking at 200 rpm between scans. sfGFP emission values were referenced against mScarlet emission values as a measure of cell density, and normalised to initial start point to show the relative change over time. Three replicates were used to generate average and standard deviation values.

The graph in FIG. 10 shows that, as salt concentration increased, the relative expression of sfGFP expression also increased, and that this change in expression could be tracked over time. This demonstrates that the DUF1646 Na⁺ sensitive riboswitch can be used to provide a bacterial response to different salt concentrations in their surrounding media.

Example 8: Measure of Fluorescence Over Time with a Heat Responsive Genetic Element (IbpA Promoter).

Two E. coli strains were produced—the control (OR2—OR1 sfGFP and OR2-OR1 mScarlet) and the EVA strain (IbpA sfGFP and OR2—OR1 mScarlet). E. coli were cultured in bulk and adhered to the surface of PCL pellets as described above. The resulting pellets were then extruded into a filament of PCL plus bacteria, and 3D printed into a structure. Manufacturing of the filament was performed by culturing E. coli to a high density before being spun down into a pellet, adding 60% PVA/LB media solution, coated onto PCL pellets, and dried in a dehydrator before being fed into an extruder to form a filament.

Values were normalised to 1 at the starting time point to show change over time with both fluorescent protein measurements on the same axis (as both use different settings and gains this is more comparable). After 2 hours, both strains were removed from the BMG Clariostar and placed on an Eppendorf Thermomixer set to 80° C. for 1 minute, the placed back into the Clariostar to continue scanning.

As seen in FIG. 11, the control strain showed a constant increase in both sfGFP and mScar as the bacteria grew, with heating having no real impact, while the IbpA_sfGFP strain did not express sfGFP until the heat shock was applied.

This experiment demonstrates that the IbpA promoter can be used to control gene expression in a temperature-dependent manner in vivo, and that heating such as this can be used as a genetic trigger.

### Example 9: Assembly and Confirmation of PCLase Encoding Plasmid.

Plasmids encoding PCLase-1 and PCLase-2 were each joined to a non-functional sfGFP using NEBuilder kit and amplified via PCR. PCR products and a pUC19 vector were then digested using HindIII and ligated together to produce a correctly assembled plasmid with a size of approximately 4.4 kb (FIG. 12a). Colony PCR of degradation experiment bacterial stocks was performed to confirm the presence of PCLase-1 within the strain being tested. The primers used for this PCR amplify outwardly from within the insert to include the entire 4.4 kb plasmid within the amplicon (FIG. 12b). This confirms that the control strain does not carry the PCLase-1 gene but the PCLase-1 strain being tested does.

Example 10: Degradation of PCL by an Engineered E. coli Strain.

Thin flakes of PCL were incubated with E. coli for 24 hours at 37° C., shaking at 200 rpm. E. coli were either pre-cultured and pelleted down to begin the experiment at a high density, or introduced at a low density to grow with the PCL flakes present. The media used was Lennox LB media optimised for both PCLase-1 activity (pH 8.5, 5 mM Mg²⁺, 5 mM Ca²⁺, 1 mM Fe (III) (Li L. et al supra)) and E. coli protein secretion (1% glycine, 5% sucrose, 0.5% Tween-80 (Bao R-M. et al supra)). E. coli were either an unmodified control strain or an engineered PCLase-1 strain. A blank, non bacterial set of replicates was also included. PCL flakes were weighed before the experiment, cultured, then washed with ethanol and left to dry before weighing again. All experiments were performed in triplicate.

As shown in FIG. 13, the blank media and control strains showed little change to PCL mass, in some instances increasing in mass (potentially due to incomplete drying). The PCL cultured with the PCLase-1 strain showed a decrease in mass, with the higher density of bacteria being used producing the largest decrease in mass.

These results demonstrate that bacteria can be engineered to degrade a specific plastic and can be done so in a modular fashion using previously established expression systems.

### Example 11: Deactivation of Spore Formation Induced by Heating, Followed by Salt Dependent Degradation of PET Plastic Pellets or Filaments.

This experiment will test a more complex bacterial system, at a high temperature, to assess long term storage of the resulting product. In particular, PET plastic pellets for use in injection moulding or PET plastic filament for use in 3D printing containing a spore forming B. subtilis with a heat shock system controlling spore formation, and a salt-dependent riboswitch controlled PETase will be investigated.

Bacteria will be cultured and induced into spore formation. Powdered PET and bacteria will then be formed into pellets or filaments for storage and future injection moulding or 3D printing, respectively, into a final product.

The heating at injection moulding or 3D printing re-activates the bacteria and brings them out of spore formation. Upon exposure to high NaCl concentrations the bacteria express PETases that then degraded the surrounding plastic material.

The experiments performed so far have successfully formed PCL 3D printer filaments containing fluorescent E. coli which have been successfully printed into solid structures in which the bacteria have remained viable. The experiments have also demonstrated salt-induced and heat-induced genetic controls in E. coli, with the salt-induced control also being demonstrated within printed plastic parts, illustrating that both the manufacture and genetic aspects of the invention are compatible. All genetic work has been performed using fluorescent reporter molecules as a quantifiable proxy for degradation enzymes, the modular aspect of the system means that sfGFP (green)/mScarlet (red) can be exchanged for a desired degradation enzyme. The use of fluorescent proxies is commonly used within biology, both as an effective measure of bacterial density and fitness (Schlechter R. O. et al (2021) Applied and Environmental Microbiology, 87 (18), e00982-21) and as a method of investigating highly complex gene expression networks far more sophisticated than those used in this project (Mehta S. and Zhang J. (2011) Ann. Rev. Biochem., 80, 375-401).

FIGS. 14, 15 and 16 illustrate designs for a “counting” mechanism using heat triggered recombinases to allow the number of heating events to be counted. FIG. 14 shows a basic plan having a single heating event that induces permanent expression of sfGFP. FIG. 15 illustrates a medium complexity plan that uses two heating events to express sfGFP permanently. FIG. 16 is an advanced plan in which a first heating event induces spore formation and a second heating event reverses spore formation and activates permanent sfGFP production.

Embodiments of the invention are set out in the following clauses:
Clause 1. An autodegradable plastic material impregnated with a genetically engineered bacteria, wherein the bacteria are engineered to include a genetic switch that is activated by an environmental factor, and wherein activation of the switch causes, enhances or increases expression of an enzyme that degrades the plastic.
Clause 2. The plastic material of clause 1, wherein the switch is either temporarily activatable or permanently activatable by the environmental factor.
Clause 3. The plastic material of clause 1 or clause 2, wherein the switch is activatable only while or once exposed to the environmental factor.
Clause 4. The plastic material of any one of clauses 1 to 3, wherein the environmental factor is a change in pH, light, temperature, microenvironment, or in the presence of ions or ligands.
Clause 5. The plastic material of any one of clauses 1 to 4, wherein the environmental factor is a change or a drop in pH.
Clause 6. The plastic material of clause 5, wherein the bacteria are engineered to include a pH-activated promoter, such as ADAR (arginine-dependent acid resistance) or GDAR (glutamate-dependent acid-resistance) promoter systems.
Clause 7. The plastic material of any one of clauses 1 to 4, wherein the environmental factor is the presence of sodium ions and/or chloride ions, an increase in salinity, a change in osmotic potential, and/or a high salinity (around 33 g/L or above) environment.
Clause 8. The plastic material of clause 7, wherein the bacteria are engineered to include a salt-activated promoter, an osmotic promoter system, or Na+-sensitive riboswitch.
Clause 9. The plastic material of any one of clauses 1 to 8, wherein the bacteria are capable of activation from, and optionally deactivation to, a dormant state.
Clause 10. The plastic material of clause 9, wherein the bacteria are capable of forming spores (sporulating).
Clause 11. The plastic material of clause 9 or clause 10, wherein the bacteria are selected from E. coli and Bacillus and Clostridium species, including B. subtilis and C. difficile.
Clause 12. The plastic material of any one of clauses 1 to 11, wherein the plastic is in the form of feedstock, filaments for use in 3D printing, beads or pellets for use in injection moulding, or sheets for use in vacuum moulding.
Clause 13. The plastic material of any one of clauses 1 to 12, wherein the plastic is a thermoplastic, such as a polycaprolactone (PCL), polythene, high-density polyethene (HDPE) or low-density polyethene (LDPE), polycarbonates, Acrylics, Polyamides, Polystyrenes, polypropylenes, Acrylonitrile Butadiene Styrenes (ABS), polyesters including polycaprolactone or PET.
Clause 14. The plastic material of any one of clauses 1 to 13, wherein the bacteria further include one or more additional genetic switches.
Clause 15. The plastic material of clause 14, wherein the one or more additional genetic switch changes the bacteria from an active to a dormant state or from a dormant to an active state.
Clause 16. The plastic material of any one of clauses 1 to 14, wherein the bacteria are present in the plastic in a dormant state and further include one or more additional genetic switches that activate the bacteria from the dormant to an active state.
Clause 17. The plastic material of clause 15 or clause 16, wherein the dormant state of the bacteria is as a spore.
Clause 18. The plastic material of any one of clauses 14 to 17, wherein the one or more additional genetic switches is a temperature sensitive genetic switch.
Clause 19. The plastic material of clause 18, wherein one or more additional genetic switches is, or is derived from, a thermo-sensitive promoter, such as a heat shock promoter, heat sensitive G-quadruplex DNA nanostructures and/or a thermosensitive riboswitch.
Clause 20. The plastic material of any one of clauses 14 to 19, wherein the one or more additional genetic switches is activated by the same or different environmental factor(s) to a first genetic switch.
Clause 21. The plastic material of any one of clauses 14 to 20, wherein activation of one genetic switch causes a different change in behaviour to activation of the one or more additional switches.
Clause 22. The plastic material of any one of clauses 14 to 21, wherein at least one of the genetic switches activates expression of an enzyme that degrades the plastic material.
Clause 23. The plastic material of any of clauses 14 to 22, wherein at least one of the genetic switches is permanently activatable by an environmental factor.
Clause 24. The plastic material of clause 23, wherein all the genetic switches are permanent switches.
Clause 25. The plastic material of any of clauses 14 to 24, wherein activation of a first genetic switch precedes activation of a second genetic switch.

It will be appreciated that the aim of the present invention is to help solve the problem of accumulation of plastic waste, while not compromising on the desired longevity and durability of plastic for numerous commercial applications. This has been achieved by the production of plastics containing bacteria genetically engineered to degrade their surrounding material in response to environmental stimuli. The thought is that, if a plastic product such as packaging has made its way into the ocean or landfill, its purpose has been fulfilled.

1. An autodegradable plastic material impregnated with a genetically engineered bacteria, wherein the bacteria are engineered to include a genetic switch that is activated by an environmental factor, and wherein activation of the switch causes, enhances or increases expression of an enzyme that degrades the plastic.

2. The plastic material of claim 1, wherein the environmental factor is a change in pH, light, temperature, microenvironment, or in the presence of ions or ligands.

3. The plastic material of claim 1, wherein the environmental factor is the presence of sodium ions and/or chloride ions, an increase in salinity, a change in osmotic potential, and/or a high salinity environment.

4. The plastic material of claim 3, wherein the bacteria are engineered to include a salt-activated promoter, an osmotic promoter system, or Na⁺-sensitive riboswitch.

5. The plastic material of claim 1, wherein the environmental factor is a change or a drop in pH.

6. The plastic material of claim 5, wherein the bacteria are engineered to include a pH-activated promoter.

7. The plastic material of claim 1, wherein the switch is either temporarily activatable or permanently activatable by the environmental factor.

8. The plastic material of claim 1, wherein the switch is activatable only while or once exposed to the environmental factor.

9. The plastic material of claim 1, wherein the bacteria are capable of activation from a dormant state.

10. The plastic material of claim 9, wherein the bacteria are capable of forming spores (sporulating).

11. The plastic material of claim 9, wherein the bacteria are selected from E. coli and Bacillus and Clostridium species.

12. The plastic material of claim 1, wherein the plastic is in the form of feedstock, filaments for use in 3D printing, beads or pellets for use in injection moulding, or sheets for use in vacuum moulding.

13. The plastic material of claim 1, wherein the plastic is a thermoplastic.

14. The plastic material of claim 1, wherein the bacteria further include one or more additional genetic switches.

15. The plastic material of claim 14, wherein the one or more additional genetic switch changes the bacteria from an active to a dormant state or from a dormant to an active state.

16. The plastic material of claim 1, wherein the bacteria are present in the plastic in a dormant state and further include one or more additional genetic switches that activate the bacteria from the dormant to an active state.

17. (canceled)

18. The plastic material of claim 14, wherein the one or more additional genetic switches is a temperature sensitive genetic switch.

19. (canceled)

20. (canceled)

21. The plastic material of claim 14, wherein activation of one genetic switch causes a different change in behaviour to activation of the one or more additional switches.

22. The plastic material of claim 14, wherein at least one of the genetic switches activates expression of an enzyme that degrades the plastic material.

23. (canceled)

24. (canceled)

25. The plastic material of claim 14, wherein activation of a first genetic switch precedes activation of a second genetic switch.
